SEQUENCE LISTING (11 sequences)

SEQ ID NO: 1 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 2 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 216 aa, synthetic construct
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 5 - protein, 445 aa, synthetic construct
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 219 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 444 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 214 aa, synthetic construct
DIQMTQSPSSLSASVGDRVTITCKASDHINNWLAWYQQKPGQAPRLLISGATSLETGVPSRFSGSGTGKDYTLTISSLQPEDFATYYCQQYWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 444 aa, synthetic construct
QVQLVQSGAELKKPGASVKLSCKASGYTFTSYGISWVKQATGQGLEWIGEIYPRSGNTYYNEKFKGRATLTADKSTSTAYMELRSLRSEDSAVYFCARSTTVRPPGIWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 214 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 450 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2024/0369467 A1
(43) Pub. Date: Nov. 7, 2024

(54) METHODS FOR TREATING PATIENTS WITH AN AUTOANTIBODY-MEDIATED DISEASE

(71) Applicant: argenx BV, Ghent (BE)

(72) Inventors: Peter Verheesen, Ghent (BE); Magdalena Sips, Ghent (BE); Robert Pollmann, Marburg (DE); Michael Hetl, Marburg (DE); Pascal Joly, Mont Saint Aignan Cedex (FR); Sebastien Calbo, Mont Saint Aignan Cedex (FR); Maud Maho-Vaillant, Mont Saint Aignan Cedex (FR)

(21) Appl. No.: 18/774,570

(22) Filed: Jul. 16, 2024

Related U.S. Application Data
(63) Continuation of application No. PCT/EP2023/050980, filed on Jan. 17, 2023.
(60) Provisional application No. 63/266,852, filed on Jan. 17, 2022.

(51) Int. Cl. G01N 15/14; A61K 31/573; A61K 39/00; A61P 37/06; C07K 16/28; G01N 15/10

(52) U.S. Cl. CPC G01N 15/14; A61K 31/573; A61P 37/06; C07K 16/283; A61K 2039/505; G01N 2015/1006; G01N 2015/1486; G01N 2800/24; G01N 2800/52; G01N 2800/54

(57) ABSTRACT

Provided herein are methods of treating an autoantibody-mediated disease in a subject, methods of monitoring treatment of an autoantibody-mediated disease in a subject, based on the frequency of B cells in the subject.

### BACKGROUND

It is estimated that more than 2.5% of the human population is affected by autoantibody-driven autoimmune diseases, in which autoreactive antibodies are directly pathogenic. Therapeutic antagonism of the neonatal Fc receptor (FcRn), a major histocompatibility complex class I-like molecule that is involved in the recycling of immunoglobulin G (IgG) and is thus responsible for the long half-life of IgG, has been explored as a strategy to treat IgG-mediated autoimmune diseases such as generalized myasthenia gravis (gMG), immune thrombocytopenia (ITP), and pemphigus (pemphigus vulgaris (PV) and pemphigus foliaceus (PF)). The remarkable clinical efficacy of FcRn antagonism appears to be directly linked to early removal of pathogenic IgG autoantibodies from circulation.

While treatment with FcRn antagonists has shown remarkable clinical efficacy in treating autoimmune diseases, there are currently no established biomarkers for measuring treatment efficacy, and no established prognostic methods for determining the risk of relapse in a patient.

Accordingly, there is a need in the art for novel biomarker-based methods of monitoring treatment efficacy and remission following FcRn antagonist treatment of patients that have an autoantibody-mediated disease.

### SUMMARY

The instant disclosure demonstrates that treatment with an FcRn antagonist causes a reduction in the frequency of circulating B cells in patients who respond to the FcRn antagonist. Further, patients who do not respond to treatment with an FcRn antagonist do not have a reduced frequency of B cells. Accordingly, provided herein are methods of monitoring treatment efficacy and remission of an autoantibody-mediated disease in a subject following treatment with an FcRn antagonist, based on the frequency of B cells in the subject. Also provided herein are methods of using an FcRn antagonist for treating an autoantibody-mediated disease in a subject that has an increased frequency of B cells and has relapsed following previous treatment with a first FcRn antagonist.

In an aspect, provided herein is a method for monitoring efficacy of treatment of an autoantibody-mediated disease in a subject following treatment with a first FcRn antagonist, the method comprising: a) measuring in vitro the frequency of B cells in a blood sample taken from the subject; and b) comparing the frequency of B cells to a reference value associated with the autoantibody-mediated disease in the subject, wherein the treatment is not effective if the frequency of B cells in the sample is greater than or equal to the reference value, and wherein the treatment is effective if the frequency of B cells is less than the reference value.

In an aspect, provided herein is a method of treating an autoantibody-mediated disease in a subject that has received a first FcRn antagonist and is receiving a corticosteroid dosing regimen, the method comprising: a) administering to the subject a therapeutically effective amount of a second FcRn antagonist; b) measuring in vitro the frequency of B cells in a blood sample taken from the subject; and c) comparing the frequency of B cells to a reference value associated with the autoantibody-mediated disease in the subject, wherein the corticosteroid dosing regimen is maintained if the frequency of B cells in the sample is greater than or equal to the reference value, or wherein corticosteroid dosing regimen is tapered if the frequency of B cells is less than the reference value.

In an aspect, provided herein is a second FcRn antagonist for use in a method of treating an autoantibody-mediated disease in a subject that has received a first FcRn antagonist and is receiving a corticosteroid dosing regimen, wherein: a) a therapeutically effective amount of the second FcRn antagonist is administered to the subject; b) the frequency of B cells in a blood sample taken from the subject is measured in vitro; and c) the frequency of B cells is compared to a reference value associated with the autoantibody-mediated disease in the subject, wherein the corticosteroid dosing regimen is maintained if the frequency of B cells in the sample is greater than or equal to the reference value, and wherein corticosteroid dosing regimen is tapered if the frequency of B cells is less than the reference value.

In an aspect, provided herein is a method for treating an autoantibody-mediated disease in a subject comprising: (a) administering to the subject one or more initial doses of a therapeutically effective amount of a first FcRn antagonist, (b) administering to the subject one or more further doses of a therapeutically effective amount of a second FcRn antagonist if the frequency of B cells in the subject after step (a) is greater than or equal to a reference value associated with the autoantibody-mediated disease in the subject, or discontinuing treatment with the first FcRn antagonist if the frequency of B cells in the subject after step (a) is less than a reference value associated with active disease in the subject.

In an aspect, provided herein is an FcRn antagonist for use in a method of treating an autoantibody-mediated disease in a subject, wherein (a) one or more initial doses of a therapeutically effective amount of a first FcRn antagonist is administered to the subject, and (b) one or more further doses of a therapeutically effective amount of a second FcRn antagonist is administered to the subject if the frequency of B cells in the subject after step (a) is greater than or equal to a reference value associated with the autoantibody-mediated disease in the subject or the first FcRn antagonist is discontinued if the frequency of B cells in the subject after step (a) is less than a reference value associated with the autoantibody-mediated disease in the subject.

In an embodiment, the therapeutically effective amount of the first FcRn antagonist is a dose of about 10 mg/kg to about 30 mg/kg, administered intravenously.

In an embodiment, the therapeutically effective amount of the first FcRn antagonist is a dose of about 750 mg to about 3000 mg, administered subcutaneously.

In an aspect, provided herein is a method for determining if a subject that has previously been treated for an autoantibody-mediated disease using a first FcRn antagonist requires further treatment with a second FcRn antagonist, the method comprising: a) measuring in vitro the frequency of B cells in a blood sample taken from the subject; and b) comparing the frequency of B cells to a reference value associated with the autoantibody-mediated disease in the subject, wherein if the frequency of B cells in the sample is greater than or equal to the reference value, then the subject is need of further treatment with the second FcRn antagonist.

In an aspect, provided herein is a method for treating an autoantibody-mediated disease in a subject comprising: administering to the subject a therapeutically effective amount of a second FcRn antagonist, wherein the autoantibody-mediated disease has relapsed in the subject following prior therapy with a first FcRn antagonist and wherein the subject has a frequency of B cells that is greater than or equal to a reference value associated with the autoantibody-mediated disease in the subject.

In an aspect, provided herein is a second FcRn antagonist for use in a method of treating an autoantibody-mediated disease in a subject, wherein the autoantibody-mediated disease has relapsed in the subject following prior therapy with a first FcRn antagonist and wherein the subject has a frequency of B cells that is greater than or equal to a reference value associated with the autoantibody-mediated disease in the subject.

In an aspect, provided herein is a method for treating an autoantibody-mediated disease in a subject comprising administering to the subject a therapeutically effective amount of a second FcRn antagonist, wherein the therapeutically effective amount of the FcRn antagonist is determined based on the frequency of B cells in a blood sample taken from the subject.

In an aspect, provided herein is a second FcRn antagonist for use in a method of treating an autoantibody-mediated disease in a subject, wherein the autoantibody-mediated disease has relapsed in the subject following prior therapy with a first FcRn antagonist, the method comprising administering to the subject a therapeutically effective amount of a second FcRn antagonist, wherein the therapeutically effective amount is determined based on the frequency of B cells in a blood sample taken from the subject.

In an aspect, provided herein is a method for monitoring remission of an autoantibody-mediated disease in a subject following treatment with a first FcRn antagonist, the method comprising: a) measuring in vitro the frequency of B cells in a blood sample taken from the subject; and b) comparing the frequency of B cells to a reference value associated with the autoantibody-mediated disease in the subject, wherein the subject is in remission from the autoantibody-mediated disease if the frequency of B cells in the sample is lower than or equal to the reference value.

In an embodiment, the corticosteroid dose regimen is tapered to a lower dose amount or a lower dosing frequency. In an embodiment, the method further comprises administering to the subject a therapeutically effective amount of the second FcRn antagonist if the frequency of B cells in the sample is greater than or equal to the reference value. In an embodiment, the method further comprises administering to the subject a therapeutically effective amount of a second FcRn antagonist if the frequency of B cells in the sample is greater than or equal to the reference value.

In an embodiment, the reference value is about 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold or 7-fold times a normal frequency of B cells. In an embodiment, the normal frequency of B cells is about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of lymphocytes. In an embodiment, the reference value is about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of lymphocytes.

In an embodiment, the reference value is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the maximum frequency of B cells measured in the subject prior to receiving any treatment for the autoantibody-mediated disease. In an embodiment, the reference value is greater than 60% of the maximum frequency of B cells measured in the subject prior to receiving any treatment for the autoantibody-mediated disease.

In an embodiment, the reference value is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% higher than the lowest frequency of B cells measured in the subject following treatment with the first FcRn antagonist for the autoantibody-mediated disease.

In an embodiment, the reference value is about 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold or 7-fold times the lowest frequency of B cells measured in the subject following treatment with the first FcRn antagonist for the autoantibody-mediated disease.

In an embodiment, the subject was previously treated with the first FcRn antagonist at a dose of about 10 mg/kg to about 30 mg/kg, administered intravenously. In an embodiment, the subject was previously treated with the first FcRn antagonist at a dose of about 750 mg to about 3000 mg, administered subcutaneously.

In an embodiment, the subject was also previously treated with a corticosteroid or an immunosuppressive agent.

In an embodiment, the effective amount of the second FcRn antagonist is a higher dose than the previous treatment with the first FcRn antagonist. In an embodiment, the effective amount of the second FcRn antagonist is a lower dose than the previous treatment with the first FcRn antagonist.

In an embodiment, the effective amount of the second FcRn antagonist is administered more frequently compared to the previous treatment with the first FcRn antagonist. In an embodiment, the effective amount of the second FcRn antagonist is administered less frequently compared to the previous treatment with the first FcRn antagonist.

In an embodiment, the effective amount of the second FcRn antagonist is administered intravenously at a dose of about 10 mg/kg to about 30 mg/kg once weekly, every two weeks, every three weeks, every four weeks, or every six weeks. In an embodiment, the effective amount of the second FcRn antagonist is administered intravenously at a dose of 10 mg/kg once weekly, every two weeks, every three weeks, every four weeks, or every six weeks. In an embodiment, the effective amount of the second FcRn antagonist is administered intravenously at a dose of 25 mg/kg once weekly, every two weeks, every three weeks, every four weeks, or every six weeks.

In an embodiment, the effective amount of the second FcRn antagonist is administered subcutaneously at a fixed dose of about 750 mg to about 3000 mg once weekly, every two weeks, every three weeks, every four weeks, or every six weeks. In an embodiment, the effective amount of the FcRn second antagonist is administered subcutaneously at a fixed dose of 1000 mg or 2000 mg once weekly, every two weeks, every three weeks, every four weeks, or every six weeks.

In an embodiment, the method further comprises administering to the subject an effective amount of a corticosteroid or an immunosuppressive agent. In an embodiment, the effective amount of the corticosteroid is administered at a dose of about 0.5 mg/kg per day. In an embodiment, the effective amount of the corticosteroid is administered at a dose of about 0.25 mg/kg per day. In an embodiment, the effective amount of the corticosteroid is administered at a dose of about 20 mg per day. In an embodiment, the effective amount of the corticosteroid is administered at a dose of about 10 mg per day.

In an embodiment, the frequency of B cells is measured with flow cytometry. In an embodiment, the B cells are CD19+ B cells.

In an embodiment, the first FcRn antagonist and the second FcRn antagonist are each the same FcRn antagonist. In an embodiment, the first FcRn antagonist and the second FcRn antagonist are each a different FcRn antagonist.

In an embodiment, the FcRn antagonist is an anti-FcRn antibody. In an embodiment, the first FcRn antagonist is an anti-FcRn antibody. In an embodiment, the second FcRn antagonist is an anti-FcRn antibody.

In an embodiment, the anti-FcRn antibody is rozanolixizumab (UCB7665), nipocalimab (M281), orilanolimab (ALXN1830/SYNT001), or batoclimab (IMVT-1401/RVT1401/HBM9161).

In an embodiment, the FcRn antagonist is an Fc region comprising amino acids Y, T, E, K, F, and Y at EU positions 252, 254, 256, 433, 434, and 436, respectively. In an embodiment, the first FcRn antagonist or the second FcRn antagonist is an Fc region comprising amino acids Y, T, E, K, F, and Y at EU positions 252, 254, 256, 433, 434, and 436, respectively.

In an embodiment, the FcRn antagonist is efgartigimod. In an embodiment, the first FcRn antagonist or the second FcRn antagonist is efgartigimod. In an embodiment, the FcRn antagonist comprises the amino acid sequence of SEQ ID NO: 1, 2, or 3. In an embodiment, the first FcRn antagonist or the second FcRn antagonist comprises the amino acid sequence of SEQ ID NO: 1, 2, or 3.

In an embodiment, the first FcRn antagonist is an anti-FcRn antibody and the second FcRn antagonist is efgartigimod. In an embodiment, the first FcRn antagonist is an anti-FcRn antibody and the second FcRn antagonist comprises the amino acid sequence of SEQ ID NO: 1, 2, or 3. In an embodiment, the anti-FcRn antibody is rozanolixizumab (UCB7665), nipocalimab (M281), orilanolimab (ALXN1830/SYNT001), or batoclimab (IMVT-1401/RVT1401/HBM9161). In an embodiment, the patient has not been previously treated with efgartigimod.

In an embodiment, the subject has a serum level of a pathogenic IgG autoantibody—that is associated with a relapse of the autoantibody-mediated disease. In an embodiment, the pathogenic IgG autoantibody is an anti-Dsg-3 antibody or an anti-Dsg-1 antibody.

In an embodiment, the autoantibody-mediated disease is selected from the group consisting of: allogenic islet graft rejection, alopecia areata, ankylosing spondylitis, antiphospholipid syndrome (APS), autoimmune Addison's disease, Alzheimer's disease, antibody-mediated allograft rejection (AMR), antineutrophil cytoplasmic autoantibodies (ANCA), ANCA vasculitis, autoimmune diseases of the adrenal gland, autoimmune encephalitis, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenia purpura or idiopathic thrombocytopenia purpura or immune-mediated thrombocytopenia), autoimmune urticaria, Behcet's disease, bullous pemphigoid (BP), cardiomyopathy, Castleman's syndrome, celiac spruce-dermatitis, chronic fatigue immune disfunction syndrome, chronic inflammatory demyelinating polyneuropathy (CIDP), Churg-Strauss syndrome, cicatricial pemphigoid, CREST syndrome, cold agglutinin disease, COVID-19 mediated postural orthostatic tachycardia syndrome (POTS), Crohn's disease, delayed graft function after kidney transplant, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre syndrome (GBS), Goodpasture's syndrome, graft-versus-host disease (GVHD), Hashimoto's thyroiditis, hemophilia A, hemolytic disease of the fetus and newborn (HDFN), idiopathic membranous neuropathy, idiopathic pulmonary fibrosis, IgA neuropathy, IgM polyneuropathies, juvenile arthritis, Kawasaki's disease, lichen planus, lichen sclerosus, lupus erythematosus, lupus nephritis, membranous neuropathy, membranous nephropathy, Ménière's disease, mixed connective tissue disease, mucous membrane pemphigoid, multiple sclerosis, Type 1 diabetes mellitus, multifocal motor neuropathy (MMN), myelin oligodendrocyte glycoprotein antibody-associated disease (MOGAD), myasthenia gravis (MG), generalized myasthenia gravis (gMG), ocular myasthenia gravis (OMG), myositis, neuromyelitis optica (NMO), paraneoplastic bullous pemphigoid, pemphigoid gestationis, pemphigus vulgaris (PV), pemphigus foliaceus (PF), pernicious anemia, polyarteritis nodosa, polychrondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Raynaud's phenomenon, Reiter's syndrome, rheumatoid arthritis (RA), sarcoidosis, scleroderma, Sjögren's syndrome, solid organ transplant rejection, stiff-man syndrome, systemic lupus erythematosus (SLE), Takayasu's arteritis, toxic epidermal necrolysis (TEN), Stevens-Johnson syndrome (SJS), temporal arteritis/giant cell arteritis, thrombotic thrombocytopenia purpura (TTP), thyroid eye disease, ulcerative colitis, uveitis, warm autoimmune hemolytic anemia (wAIHA), dermatitis herpetiformis vasculitis, anti-neutrophil cytoplasmic antibody-associated vasculitides, vitiligo, and Wegner's granulomatosis.

In an embodiment, the autoantibody-mediated disease is pemphigus vulgaris (PV). In an embodiment, the autoantibody-mediated disease is pemphigus foliaceus (PF).

In an embodiment, the subject has one or more physical symptoms of an autoantibody-mediated disease following treatment with the first FcRn antagonist. In an embodiment, the one or more physical symptoms include but are not limited to ocular muscle fatigue or weakness, skeletal muscle fatigue or weakness, respiratory muscle fatigue or weakness, disabling fatigue, slurred speech, choking, impaired swallowing, double or blurred vision, immobility requiring assistance, shortness of breath, respiratory failure, and blisters, including skin and mouth blisters.

### BRIEF DESCRIPTION OF THE DRAWINGS

FIG. 1 is a series of FACS plots showing the gating strategy used to study CD19+ IgM− IgD− DSG3+ or Col7+ cells for one representative patient.

FIG. 2A is a bar graph showing clinical responses to efgartigimod treatment for all patients in cohorts 3 and 4. Arrows indicate when efgartigimod was administered; the dotted line indicates every other week administration time period in patients who achieved EoC. FIG. 2B is a table showing symbol shapes used throughout the Figures for selected patients. FIG. 2C is a graph showing levels of pathogenic (anti-Dsg-3) and non-pathogenic antibodies (anti-varicella zoster virus (VZV); anti-tetanus toxoid (TT); and anti-pneumococcal capsular polysaccharide (PCP)) as well as total IgG (tIgG) and PDAI (pemphigus disease area index) activity score in one representative PV patient. FIG. 2D is a graph showing levels of pathogenic (anti-Dsg-1) and non-pathogenic antibodies (anti-VZV; anti-TT; and anti-PCP) as well as tIgG and PDAI activity score in one representative PF patient. The gray shaded area indicates efgartigimod treatment-free follow up period. Abbreviations: EoT, end of treatment; DC, disease control; EoC, end of consolidation; CR, complete clinical remission; CRmin, complete clinical remission with minimal treatment; Pt, patient.

FIGS. 3A-F are graphs showing levels of anti-Dsg IgG subtypes in patients with pemphigus at baseline, complete clinical remission (CR), end of treatment (EoT), and end of study (EoS). FIG. 3A is a graph showing anti-Dsg3 IgG1 levels in patients with PV.

FIG. 3B is a graph showing anti-Dsg3 IgG2 levels in patients with PV. FIG. 3C is a graph showing anti-Dsg3 IgG3 levels in patients with PV. FIG. 3D is a graph showing anti-Dsg3 IgG4 levels in patients with PV. Dotted lines in FIGS. 3A-3D indicate the threshold of positivity (2 SD above mean value from 36 healthy donors). FIG. 3E is a graph showing anti-Dsg1 IgG1 levels in patients with PF. FIG. 3F is a graph showing anti-Dsg1 IgG4 levels in patients with PF. Dotted lines in FIGS. 3E-3F indicate the threshold of positivity (20 RU/mL).

FIGS. 4A-B are graphs showing levels of circulating IgG immune complexes (IgG CIC) in patients with PV (FIG. 4A) and PF (FIG. 4B) following efgartigimod treatment at baseline, CR, EoT, and EoS. Dotted line indicates clinical significance (CS). Abbreviation: CIC, circulating immune complexes.

FIG. 5A is a series of representative flow cytometry plots showing the frequency of Dsg-3+ switched memory B cells (MBC) at baseline, CR, and EoT for one patient with PV. FIGS. 5B-5C are graphs showing the frequency of circulating Dsg-3+ switched memory B cells in three patients with PV during the study in relation to Dsg-3 autoantibody serum titers (FIG. 5B) and by time point (FIG. 5C). FIGS. 5D-5E are graphs showing antibody titer of anti-Dsg-3 antibodies (FIG. 5D) and frequency of circulating Dsg-3+ switched memory B cells in five patients with PV (FIG. 5E) (3 with sustained clinical response shown in FIGS. 5B-5C, and 2 with relapse following CR). FIG. 5F is a series of representative ELISPOT assays measuring total IgG-ASCs (antibody secreting cells) with 2.5×1⁰³ to 1×10⁴ PBMCs plated per well (top), and Dsg-1 IgG ASCs with 1×10⁵ to 4×10⁵ PBMCs plated per well (bottom), detected in PBMCs from a patient with PF at baseline (BL) and end of study (EoS). FIGS. 5G-H are graphs showing the frequency of peripheral blood Dsg-1-specific ASCs evaluated by ELISPOT assay in 3 patients with PF during the study both in relation to Dsg-1 autoantibody serum titers (FIG. 5G) and by time point (FIG. 5H). Frequencies are reported as a percentage of total IgG ASC.

FIGS. 6A-6E are graphs showing frequencies of lymphocyte subsets in patients with pemphigus. FIG. 6A is a graph showing the frequency of leukocytes in PBMCs of patients with pemphigus in the efgartigimod study. Dotted lines represent the normal range of 3.9-12.7×10⁹/L leukocytes. FIG. 6B is a graph showing the frequency of neutrophils, lymphocytes, and monocytes in PBMCs of patients with pemphigus in the efgartigimod study.

FIG. 6C is a bar graph showing the frequency of CD4+ T cells and T cell subsets. FIG. 6D is a bar graph showing the frequency of CD19+ B cells and B cell subsets. Arrows and lines indicate how the frequency of a parent population was determined. FIG. 6E is pair of graphs showing frequency and counts of CD19+ B cells at baseline, EoT, and EoS in the 9 patients from cohort 4 with sustained clinical response. The horizontal bar in each column represents the median. Dotted lines in the graph at left represent the normal range of 5-22% CD19+ B cells. Dotted lines in the graph at right represent the normal range of 80-616 CD19+ B cells/μl. A non-parametric one-way ANOVA with Dunn's post-test was performed. *: p<0.05; **: p<0.01. Abbreviation: IQR, interquartile range.

FIG. 7 is a graph showing the frequency of CD19+ B cells in patients with PV and PF with sustained clinical response, those relapsing following CR, and those with no clinical response during the study. Dotted lines in the graph represent the normal range of 5-22% CD19+ B cells.

FIG. 8A and FIG. 8B are graphs showing summary profiles of PV patients (FIG. 8A) and PF patients (FIG. 8B) patients achieving sustained clinical response, depicting prednisone dosages, PDAI activity scores, and frequency of total CD19+ B cells in peripheral blood when available. Gray shaded zone indicates efgartigimod-free follow up period.

### DETAILED DESCRIPTION

The instant disclosure demonstrates that treatment with an FcRn antagonist causes a reduction in the frequency of B cells in patients who respond to the FcRn antagonist. Further, patients who do not respond to treatment with an FcRn antagonist do not have a reduced frequency of B cells. Accordingly, provided herein are methods of monitoring remission and monitoring treatment efficacy of an autoantibody-mediated disease in a subject following treatment with an FcRn antagonist, based on the frequency of B cells in the subject. Also provided herein are methods of using an FcRn antagonist for treating an autoantibody-mediated disease in a subject that has an increased frequency of B cells and has relapsed following previous treatment with a first FcRn antagonist.

In an aspect, provided herein is a method for monitoring treatment efficacy in a subject following treatment with a first FcRn antagonist, wherein the subject has an autoantibody-mediated disease, the method comprising: a) measuring in vitro the frequency of B cells in a blood sample taken from the subject; and b) comparing the frequency of the B cells to a reference value associated with the autoantibody-mediated disease in the subject, wherein the treatment is not effective if the frequency of B cells in the sample is greater than or equal to the reference value, or wherein the treatment is effective if the frequency of B cells is less than the reference value.

In an aspect, provided herein is a method of treating an autoantibody-mediated disease in a subject that has received a first FcRn antagonist and is receiving a corticosteroid dosing regimen, the method comprising: a) administering to the subject a therapeutically effective amount of a second FcRn antagonist; b) measuring in vitro the frequency of B cells in a blood sample taken from the subject; and c) comparing the frequency of B cells to a reference value associated with the autoantibody-mediated disease in the subject, wherein the corticosteroid dosing regimen is maintained if the frequency of B cells in the sample is greater than or equal to the reference value, or wherein corticosteroid dosing regimen is tapered if the frequency of B cells is less than the reference value.

In an aspect, provided herein is a second FcRn antagonist for use in a method of treating an autoantibody-mediated disease in a subject that has received a first FcRn antagonist and is receiving a corticosteroid dosing regimen, wherein: a) a therapeutically effective amount of the second FcRn antagonist is administered to the subject; b) the frequency of B cells in a blood sample taken from the subject is measured in vitro; and c) the frequency of B cells is compared to a reference value associated with the autoantibody-mediated disease in the subject, wherein the corticosteroid dosing regimen is maintained if the frequency of B cells in the sample is greater than or equal to the reference value, and wherein corticosteroid dosing regimen is tapered if the frequency of B cells is less than the reference value.

In an aspect, provided herein is a method for treating an autoantibody-mediated disease in a subject comprising: (a) administering to the subject one or more initial doses of a therapeutically effective amount of a first FcRn antagonist, (b) administering to the subject one or more further doses of a therapeutically effective amount of a second FcRn antagonist if the frequency of B cells in the subject after step (a) is greater than or equal to a reference value associated with the autoantibody-mediated disease in the subject, or discontinuing treatment with the first FcRn antagonist if the frequency of B cells in the subject after step (a) is less than a reference value associated with active disease in the subject.

In an aspect, provided herein is an FcRn antagonist for use in a method of treating an autoantibody-mediated disease in a subject, wherein (a) one or more initial doses of a therapeutically effective amount of a first FcRn antagonist is administered to the subject, and (b) one or more further doses of a therapeutically effective amount of a second FcRn antagonist is administered to the subject if the frequency of B cells in the subject after step (a) is greater than or equal to a reference value associated with the autoantibody-mediated disease in the subject or the first FcRn antagonist is discontinued if the frequency of B cells in the subject after step (a) is less than a reference value associated with the autoantibody-mediated disease in the subject.

In an aspect, provided herein is a method for determining if a subject that has previously been treated for an autoantibody-mediated disease using a first FcRn antagonist requires further treatment with a second FcRn antagonist, the method comprising: a) measuring in vitro the frequency of B cells in a blood sample taken from the subject; and b) comparing the frequency of the B cells to a reference value associated with the autoantibody-mediated disease in the subject, wherein if the frequency of B cells in the sample is greater than or equal to the reference value, then the subject is need of further treatment with the second FcRn antagonist.

Also provided herein is a method for treating an autoantibody-mediated disease in a subject comprising: administering to the subject a therapeutically effective amount of a second FcRn antagonist, wherein the autoantibody-mediated disease has relapsed in the subject following prior therapy with a first FcRn antagonist and wherein the subject has a frequency of B cells that is greater than or equal to a reference value associated with the autoantibody-mediated disease in the subject.

Also provided herein is a method for treating an autoantibody-mediated disease in a subject comprising administering to the subject a therapeutically effective amount of a second FcRn antagonist, wherein the therapeutically effective amount of the FcRn antagonist is determined based on the frequency of B cells in a blood sample taken from the subject.

Further provided herein is a method for monitoring remission of an autoantibody-mediated disease in a subject following treatment with a first FcRn antagonist, the method comprising: a) measuring in vitro the frequency of B cells in a blood sample taken from the subject; and b) comparing the frequency of the B cells to a reference value associated with the autoantibody-mediated disease in the subject, wherein the subject is in remission from the autoantibody-mediated disease if the frequency of B cells in the sample is lower than or equal to the reference value.

### Definitions

As used herein, the term “FcRn” refers to a neonatal Fc receptor. Exemplary FcRn molecules include human FcRn encoded by the FCGRT gene as set forth in RefSeq NM 004107. The amino acid sequence of the corresponding protein is set forth in RefSeq NP_004098.

As used herein, the term “FcRn antagonist” refers to any agent that binds specifically to FcRn and inhibits the binding of immunoglobulin to FcRn (e.g., human FcRn). In an embodiment, the FcRn antagonist is an Fc region (e.g., a variant Fc region disclosed herein) that specifically binds to FcRn through the Fc region and inhibits the binding of immunoglobulin to FcRn. In an embodiment, the FcRn antagonist is not a full-length IgG antibody. In an embodiment, the FcRn antagonist comprises an antigen binding site that binds a target antigen and a variant Fc region. In an embodiment, the FcRn antagonist is an Fc fragment comprising or consisting of an Fc region and lacking an antigen binding site. In an embodiment the term “FcRn antagonist” refers to an antibody or antigen-binding fragment thereof that specifically binds to FcRn via its antigen binding domain or via its Fc region and inhibits the binding of the Fc region of immunoglobulin (e.g., IgG autoantibodies) to FcRn.

As used herein, the term “Fc domain” refers to the portion of a single immunoglobulin heavy chain beginning in the hinge region and ending at the C-terminus of the antibody. Accordingly, a complete Fc domain comprises at least a portion of a hinge (e.g., upper, middle, and/or lower hinge region) domain, a CH2 domain, and a CH3 domain.

As used herein, the term “Fc region” refers to the portion of a native immunoglobulin formed by the Fc domains of its two heavy chains. A native Fc region is homodimeric.

As used herein, the term “variant Fc region” refers to an Fc region with one or more alteration(s) relative to a native Fc region. Alteration can include amino acid substitutions, additions and/or deletions, linkage of additional moieties, and/or alteration the native glycans. The term encompasses heterodimeric Fc regions where each of the constituent Fc domains is different. The term also encompasses single chain Fc regions where the constituent Fc domains are linked together by a linker moiety.

As used herein the term “FcRn binding fragment” refers to a portion of an Fc region that is sufficient to confer FcRn binding.

As used herein, the term “EU position” refers to the amino acid position in the EU numbering convention for the Fc region described in Edelman, G. M. et al., Proc. Natl. Acad. USA, 63, 78-85 (1969) and Rabat et al, in “Sequences of Proteins of Immunological Interest”, U.S. Dept. Health and Human Services, 5th edition, 1991.

As used herein, the term “baseline” refers to a measurement (e.g., a frequency of B cells, IgG levels) in a patient, e.g., in a patient's blood, prior to the first administration (e.g., intravenous, or subcutaneous administration) of a treatment (e.g., an FcRn antagonist).

As used herein, the term “autoantibody-mediated disease” refers to any disease or disorder in which the underlying pathology is caused, at least in part, by pathogenic IgG autoantibodies.

As used herein, the term “frequency of B cells” refers to the percent of B cells in a patient's total peripheral blood mononuclear cell (PBMC) population.

As used herein, the term “treat,” “treating,” and “treatment” refer to therapeutic or preventative measures described herein. The methods of “treatment” employ administration of a polypeptide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “therapeutically effective amount” in the context of the administration of a therapy to a subject refers to the amount of a therapy that achieves a desired prophylactic or therapeutic effect.

As used herein, the term “remission” refers to a patient who has no new markers of an autoantibody-mediated disease and the baseline markers of the disease have completely resolved or healed. In an embodiment, a pemphigus patient in clinical remission has no new lesions and all established lesions are completely healed.

As used herein, the term “relapse” or “flare” refers to a patient with an autoantibody-mediated disease who has an appearance of physical symptoms and/or an increase of a marker of the autoantibody-mediated disease after a period of remission of the autoantibody-mediated disease. In an embodiment, a relapse of pemphigus refers to the appearance of at least 3 new pemphigus lesions in a 4-week period that do not heal within a week, or extension of established lesions.

As used herein, the term “subject” includes any human or non-human animal. In an embodiment, the subject is a human or non-human mammal. In an embodiment, the subject is a human.

As used herein, the term “about” or “approximately” when referring to a measurable value, such as a dosage, encompasses variations of +20%, +15%, +10%, +5%, +1%, or +0.1% of a given value or range, as are appropriate to perform the methods disclosed herein.

### FcRn Antagonists

FcRn antagonists that are useful in the methods and uses provided herein include but are not limited to any anti-FcRn antibody or any variant Fc region.

Any Fc region can be altered to produce a variant Fc region for use in the methods disclosed herein. In general, an Fc region, or FcRn-binding fragment thereof, is from a human immunoglobulin. It is understood, however, that the Fc region may be derived from an immunoglobulin of any other mammalian species, including for example, a Camelid species, a rodent (e.g., a mouse, rat, rabbit, guinea pig) or non-human primate (e.g., chimpanzee, macaque) species. Moreover, the Fc region or portion thereof may be derived from any immunoglobulin class, including IgM, IgG, IgD, IgA, and IgE, and any immunoglobulin isotype, including IgG1, IgG2, IgG3 and IgG4. In an embodiment, the Fc region is an IgG Fc region (e.g., a human IgG region). In an embodiment, the Fc region is an IgG1 Fc region (e.g., a human IgG1 region). In an embodiment, the Fc region is a chimeric Fc region comprising portions of several different Fc regions. Suitable examples of chimeric Fc regions are set forth in US 2011/0243966A1, which is incorporated herein by reference in its entirety. A variety of Fc region gene sequences (e.g., human constant region gene sequences) are available in the form of publicly accessible deposits.

An Fc region can be further truncated or internally deleted to produce a minimal FcRn-binding fragment thereof. The ability of an Fc-region fragment to bind to FcRn can be determined using any art recognized binding assay e.g., ELISA.

To enhance the manufacturability of the FcRn antagonists disclosed herein, it is preferable that the constituent Fc regions do not comprise any non-disulfide bonded cysteine residues. Accordingly, in an embodiment, the Fc regions do not comprise a free cysteine residue.

Any Fc variant, or FcRn-binding fragment thereof, that binds specifically to FcRn with increased affinity and reduced pH dependence relative to the native Fc region can be used in the methods disclosed herein. In an embodiment, the variant Fc region comprises amino acid alterations, substitutions, insertions and/or deletions that confer the desired characteristics. In an embodiment, the biologic comprises or consists of a variant Fc region, or FcRn binding fragment thereof, which binds to FcRn with a higher affinity at pH5.5 as compared to a corresponding wild-type Fc region.

In an embodiment, the variant Fc region, or FcRn binding fragment thereof consists of two Fc domains. In an embodiment, the FcRn antagonist is an Fc region comprising amino acids Y, T, E, K, F, and Y at EU positions 252, 254, 256, 433, 434, and 436, respectively.

In an embodiment, the amino acid sequence of the Fc domains of the variant Fc region comprises the amino acid sequence of SEQ ID NO: 1. In an embodiment, the amino acid sequence of the Fc domains of the variant Fc region consists of the amino acid sequence of SEQ ID NO: 1. In an embodiment, the amino acid sequence of the Fc domains of the variant Fc region comprises the amino acid sequence of SEQ ID NO: 2. In an embodiment, the amino acid sequence of the Fc domains of the variant Fc region consists of the amino acid sequence of SEQ ID NO: 2. In an embodiment, the amino acid sequence of the Fc domains of the variant Fc region comprises the amino acid sequence of SEQ ID NO: 3. In an embodiment, the amino acid sequence of the Fc domains of the variant Fc region consists of the amino acid sequence of SEQ ID NO: 3.

In an embodiment, the isolated FcRn antagonist consists of a variant Fc region, wherein the variant Fc region consists of two Fc domains which form a homodimer, wherein the amino acid sequence of each of the Fc domains consists of SEQ ID NO: 1.

In an embodiment, the isolated FcRn antagonist consists of a variant Fc region, wherein the variant Fc region consists of two Fc domains which form a homodimer, wherein the amino acid sequence of each of the Fc domains consists of SEQ ID NO: 2.

In an embodiment, the isolated FcRn antagonist consists of a variant Fc region, wherein the variant Fc region consists of two Fc domains which form a homodimer, wherein the amino acid sequence of each of the Fc domains consists of SEQ ID NO: 3.

In an embodiment, the biologic is efgartigimod (CAS Registry No. 1821402-21-4).

TABLE 1
Amino acid sequences of variant Fc regions

| SEQ ID NO: | Amino Acid Sequence |
|---|---|
| 1 | CPPCPAPELLGGPSVFLFPPKPKDTLYITREPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSRDELTKN |
| | QVSLTCLVKGFYPSDIAVEWESNGQPENNYKTTPPVLDSDGSFFLY |
| | SKLTVDKSRWQQGNVFSCSVMHEALKFHYTQKSLSLSPG |
| 2 | DKTHTCPPCPAPELLGGPSVFLFPPKPKDTLYITREPEVTCVVVDVS |
| | HEDPEVKFNWYVDGVEVHNAKTKPREEQYNSTYRVVSVLTVLHQ |
| | DWLNGKEYKCKVSNKALPAPIEKTISKAKGQPREPQVYTLPPSRDE |
| | LTKNQVSLTCLVKGFYPSDIAVEWESNGQPENNYKTTPPVLDSDGS |
| | FFLYSKLTVDKSRWQQGNVFSCSVMHEALKFHYTQKSLSLSPGK |
| 3 | DKTHTCPPCPAPELLGGPSVFLFPPKPKDTLYITREPEVTCVVVDVS |
| | HEDPEVKFNWYVDGVEVHNAKTKPREEQYNSTYRVVSVLTVLHQ |
| | DWLNGKEYKCKVSNKALPAPIEKTISKAKGQPREPQVYTLPPSRDE |
| | LTKNQVSLTCLVKGFYPSDIAVEWESNGQPENNYKTTPPVLDSDGS |
| | FFLYSKLTVDKSRWQQGNVFSCSVMHEALKFHYTQKSLSLSPG |

In an embodiment, the anti-FcRn antibody is rozanolixizumab (UCB7665), nipocalimab (M281), orilanolimab (ALXN1830/SYNT001), batoclimab (IMVT-1401/RVT1401/HBM9161).

In an embodiment, an antibody that binds specifically to FcRn and inhibits the binding of the Fc region of immunoglobulin to FcRn is nipocalimab, also known as M281. Nipocalimab is a full-length “Fc dead” IgG1 monoclonal antibody. Nipocalimab has been administered as an intravenous infusion in Phase 2 clinical trials for the treatment of myasthenia gravis (MG), warm autoimmune hemolytic anemia (WAIHA), and hemolytic disease of fetus and newborn (HDFN). Nipocalimab comprises the light chain (SEQ ID NO:4) and heavy chain (SEQ ID NO:5) sequences set forth in Table 2 below:

TABLE 2
Heavy chain and light chain sequences of nipocalimab

| SEQ ID NO: | Amino Acid Sequence |
|---|---|
| 4 | QSALTQPASVSGSPGQSITISCTGTGSDVGSYNLVSWYQQHPGKAP |
| | KLMIYGDSERPSGVSNRFSGSKSGNTASLTISGLQAEDEADYYCSSY |
| | AGSGIYVFGTGTKVTVLGQPKAAPSVTLFPPSSEELQANKATLVCLI |
| | SDFYPGAVTVAWKADSSPVKAGVETTTPSKQSNNKYAASSYLSLT |
| | PEQWKSHKSYSCQVTHEGSTVEKTVAPTECS |
| 5 | EVQLLESGGGLVQPGGSLRLSCAASGFTFSTYAMGWVRQAPGKGL |
| | EWVSSIGASGSQTRYADSVKGRFTISRDNSKNTLYLQMNSLRAEDT |
| | AVYYCARLAIGDSYWGQGTMVTVSSASTKGPSVFPLAPSSKSTSGG |
| | TAALGCLVKDYFPEPVTVSWNSGALTSGVHTFPAVLQSSGLYSLSS |
| | VVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFN |
| | WYVDGVEVHNAKTKPREEQYASTYRVVSVLTVLHQDWLNGKEY |
| | KCKVSNKALPAPIEKTISKAKGQPREPQVYTLPPSREEMTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPG |

In an embodiment, an antibody that binds specifically to FcRn and inhibits the binding of the Fc region of immunoglobulin to FcRn is rozanolixizumab, also known as UCB 7665. Rozanolixizumab is a full-length humanized IgG4 monoclonal antibody. Rozanolixizumab has been administered as a subcutaneous infusion in ongoing clinical trials for MG, immune thrombocytopenia (FTP), and chronic inflammatory demyelinating polyneuropathy (CIDP). Rozanolixizumab comprises the light chain (SEQ ID NO: 6) and heavy chain (SEQ ID NO: 7) sequences set forth in Table 3 below:

TABLE 3
Heavy chain and light chain sequences of rozanolixizumab

| SEQ ID NO: | Amino Acid Sequence |
|---|---|
| 6 | DIQMTQSPSSLSASVGDRVTITCKSSQSLVGASGKTYLYWLFQKPG |
| | KAPKRLIYLVSTLDSGIPSRFSGSGSGTEFTLTISSLQPEDFATYYCLQ |
| | GTHFPHTFGQGTKLEIKRTVAAPSVFIFPPSDEQLKSGTASVVCLLN |
| | NFYPREAKVQWKVDNALQSGNSQESVTEQDSKDSTYSLSSTLTLS |
| | KADYEKHKVYACEVTHQGLSSPVTKSFNRGEC |
| 7 | EVPLVESGGGLVQPGGSLRLSCAVSGFTFSNYGMVWVRQAPGKGL |
| | EWVAYIDSDGDNTYYRDSVKGRFTISRDNAKSSLYLQMNSLRAED |
| | TAVYYCTTGIVRPFLYWGQGTLVTVSSASTKGPSVFPLAPCSRSTSE |
| | STAALGCLVKDYFPEPVTVSWNSGALTSGVHTFPAVLQSSGLYSLS |
| | SVVTVPSSSLGTKTYTCNVDHKPSNTKVDKRVESKYGPPCPPCPAP |
| | EFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCK |
| | VSNKGLPSSIEKTISKAKGQPREPQVYTLPPSQEEMTKNQVSLTCLV |
| | KGFYPSDIAVEWESNGQPENNYKTTPPVLDSDGSFFLYSRLTVDKS |
| | RWQEGNVFSCSVMHEALHNHYTQKSLSLSLGK |

In an embodiment, an antibody that binds specifically to FcRn and inhibits the binding of the Fc region of immunoglobulin to FcRn is orilanolimab, also known as SYNT001. Orilanolimab is another full-length humanized IgG4 monoclonal antibody. Orilanolimab has been administered as an intravenous infusion in Phase 2 clinical trials for treatment of WAIHA. Orilanolimab comprises the light chain (SEQ ID NO: 8) and heavy chain (SEQ ID NO: 9) sequences set forth in Table 4 below:

TABLE 4
Heavy chain and light chain sequences of orilanolimab

| SEQ ID NO: | Amino Acid Sequence |
|---|---|
| 8 | DIQMTQSPSSLSASVGDRVTITCKASDHINNWLAWYQQKPGQAPR |
| | LLISGATSLETGVPSRFSGSGTGKDYTLTISSLQPEDFATYYCQQYW |
| | STPYTFGGGTKVEIKRTVAAPSVFIFPPSDEQLKSGTASVVCLLNNF |
| | YPREAKVQWKVDNALQSGNSQESVTEQDSKDSTYSLSSTLTLSKA |
| | DYEKHKVYACEVTHQGLSSPVTKSFNRGEC |
| 9 | QVQLVQSGAELKKPGASVKLSCKASGYTFTSYGISWVKQATGQGL |
| | EWIGEIYPRSGNTYYNEKFKGRATLTADKSTSTAYMELRSLRSEDS |
| | AVYFCARSTTVRPPGIWGTGTTVTVSSASTKGPSVFPLAPCSRSTSE |
| | STAALGCLVKDYFPEPVTVSWNSGALTSGVHTFPAVLQSSGLYSLS |
| | SVVTVPSSSLGTKTYTCNVDHKPSNTKVDKRVESKYGPPCPPCPAP |
| | EFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCK |
| | VSNKGLPSSIEKTISKAKGQPREPQVYTLPPSQEEMTKNQVSLTCLV |
| | KGFYPSDIAVEWESNGQPENNYKTTPPVLDSDGSFFLYSRLTVDKS |
| | RWQEGNVFSCSVMEALHNHYTQKSLSLSLG |

In an embodiment, an antibody that binds specifically to FcRn and inhibits the binding of the Fc region of immunoglobulin to FcRn is batoclimab, also known as IMVT1401/RVT1401/HBM9161. Batoclimab is another full-length “Fc dead” IgG1 monoclonal antibody. Batoclimab has been administered as a subcutaneous injection in ongoing Phase 2 clinical trials for treatment of MG and Graves' ophthalmopathy. Batoclimab comprises the light chain (SEQ ID NO: 10) and heavy chain (SEQ ID NO: 11) sequences set forth in Table 5 below:

TABLE 5
Heavy chain and light chain sequences of batoclimab

| SEQ ID NO: | Amino Acid Sequence |
|---|---|
| 10 | SYVLTQSPSVSVAPGQTARITCGGNNIGSKSVHWYQQKPGQAPVL |
| | VVYDDSDRPSGIPERFSASNSGNTATLTISRVEAGDEADYYCQVWD |
| | SSSDHVVFGGGTKLTVLGQPKAAPSVTLFPPSSEELQANKATLVCLI |
| | SDFYPGAVTVAWKADSSPVKAGVETTTPSKQSNNKYAASSYLSLT |
| | PEQWKSHRSYSCQVTHEGSTVEKTVAPTECS |
| 11 | QLLLQESGPGLVKPSETLSLTCTVSGGSLSSSFSYWVWIRQPPGKGL |
| | EWIGTIYYSGNTYYNPSLKSRLTISVDTSKNHFSLKLSSVTAADTAV |
| | YYCARRAGILTGYLDSWGQGTLVTVSSASTKGPSVFPLAPSSKSTS |
| | GGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCP |
| | PCPAPEAAGGPSVFLFPPKPKDTLMISRTPEVTCVVVDVSHEDPEVK |
| | FNWYVDGVEVHNAKTKPREEQYNSTYRVVSVLTVLHQDWLNGK |
| | EYKCKVSNKALPAPIEKTISKAKGQPREPQVYTLPPSREEMTKNQV |
| | SLTCLVKGFYPSDIAVEWESNGQPENNYKTTPPVLDSDGSFFLYSK |
| | LTVDKSRWQQGNVFSCSVMHEALHNHYTQKSLSLSPG |

### Methods of Use

In an aspect, provided herein is a method for monitoring efficacy of treatment of an autoantibody-mediated disease in a subject following treatment with a first FcRn antagonist, the method comprising: a) measuring in vitro the frequency of B cells in a blood sample taken from the subject; and b) comparing the frequency of B cells to a reference value associated with the autoantibody-mediated disease in the subject, wherein the treatment is not effective if the frequency of B cells in the sample is greater than or equal to the reference value, and wherein the treatment is effective if the frequency of B cells is less than the reference value.

In an aspect, provided herein is a method of treating an autoantibody-mediated disease in a subject that has received a first FcRn antagonist and is receiving a corticosteroid dosing regimen, the method comprising: a) administering to the subject a therapeutically effective amount of a second FcRn antagonist; b) measuring in vitro the frequency of B cells in a blood sample taken from the subject; and c) comparing the frequency of B cells to a reference value associated with the autoantibody-mediated disease in the subject, wherein the corticosteroid dosing regimen is maintained if the frequency of B cells in the sample is greater than or equal to the reference value, or wherein corticosteroid dosing regimen is tapered if the frequency of B cells is less than the reference value.

In an aspect, provided herein is a second FcRn antagonist for use in a method of treating an autoantibody-mediated disease in a subject that has received a first FcRn antagonist and is receiving a corticosteroid dosing regimen, wherein: a) a therapeutically effective amount of the second FcRn antagonist is administered to the subject; b) the frequency of B cells in a blood sample taken from the subject is measured in vitro; and c) the frequency of B cells is compared to a reference value associated with the autoantibody-mediated disease in the subject, wherein the corticosteroid dosing regimen is maintained if the frequency of B cells in the sample is greater than or equal to the reference value, and wherein corticosteroid dosing regimen is tapered if the frequency of B cells is less than the reference value.

In an aspect, provided herein is a method for treating an autoantibody-mediated disease in a subject comprising: (a) administering to the subject one or more initial doses of a therapeutically effective amount of a first FcRn antagonist, (b) administering to the subject one or more further doses of a therapeutically effective amount of a second FcRn antagonist if the frequency of B cells in the subject after step (a) is greater than or equal to a reference value associated with the autoantibody-mediated disease in the subject, or discontinuing treatment with the first FcRn antagonist if the frequency of B cells in the subject after step (a) is less than a reference value associated with active disease in the subject.

In an aspect, provided herein is an FcRn antagonist for use in a method of treating an autoantibody-mediated disease in a subject, wherein (a) one or more initial doses of a therapeutically effective amount of a first FcRn antagonist is administered to the subject, and (b) one or more further doses of a therapeutically effective amount of a second FcRn antagonist is administered to the subject if the frequency of B cells in the subject after step (a) is greater than or equal to a reference value associated with the autoantibody-mediated disease in the subject or the first FcRn antagonist is discontinued if the frequency of B cells in the subject after step (a) is less than a reference value associated with the autoantibody-mediated disease in the subject.

In an embodiment, the therapeutically effective amount of the first FcRn antagonist is a dose of about 10 mg/kg to about 30 mg/kg, administered intravenously.

In an embodiment, the therapeutically effective amount of the first FcRn antagonist is a dose of about 750 mg to about 3000 mg, administered subcutaneously.

In an aspect, provided herein is a method for determining if a subject that has previously been treated for an autoantibody-mediated disease using a first FcRn antagonist requires further treatment with a second FcRn antagonist, the method comprising: a) measuring in vitro the frequency of B cells in a blood sample taken from the subject; and b) comparing the frequency of the B cells to a reference value associated with the autoantibody-mediated disease in the subject, wherein if the frequency of B cells in the sample is greater than or equal to the reference value, then the subject is need of further treatment with the second FcRn antagonist.

In an aspect, provided herein is a method for treating an autoantibody-mediated disease in a subject comprising: administering to the subject a therapeutically effective amount of a second FcRn antagonist, wherein the autoantibody-mediated disease has relapsed in the subject following prior therapy with a first FcRn antagonist and wherein the subject has a frequency of B cells that is greater than or equal to a reference value associated with the autoantibody-mediated disease in the subject.

In an aspect, provided herein is a second FcRn antagonist for use in a method of treating an autoantibody-mediated disease in a subject, wherein the autoantibody-mediated disease has relapsed in the subject following prior therapy with a first FcRn antagonist and wherein the subject has a frequency of B cells that is greater than or equal to a reference value associated with the autoantibody-mediated disease in the subject.

In an aspect, provided herein is a method for treating an autoantibody-mediated disease in a subject comprising administering to the subject a therapeutically effective amount of a second FcRn antagonist, wherein the therapeutically effective amount of the FcRn antagonist is determined based on the frequency of B cells in a blood sample taken from the subject.

In an aspect, provided herein is a second FcRn antagonist for use in a method of treating an autoantibody-mediated disease in a subject, wherein the autoantibody-mediated disease has relapsed in the subject following prior therapy with a first FcRn antagonist, the method comprising administering to the subject a therapeutically effective amount of a second FcRn antagonist, wherein the therapeutically effective amount is determined based on the frequency of B cells in a blood sample taken from the subject.

In an aspect, provided herein is a method for monitoring treatment efficacy in a subject following treatment with a first FcRn antagonist, wherein the subject has an autoantibody-mediated disease, the method comprising: a) measuring in vitro the frequency of B cells in a blood sample taken from the subject; and b) comparing the frequency of the B cells to a reference value associated with the autoantibody-mediated disease in the subject, wherein the treatment is not effective if the frequency of B cells in the sample is greater than or equal to the reference value, or wherein the treatment is effective if the frequency of B cells is less than the reference value.

In an aspect, provided herein is a method for monitoring remission of an autoantibody-mediated disease in a subject following treatment with a first FcRn antagonist, the method comprising: a) measuring in vitro the frequency of B cells in a blood sample taken from the subject; and b) comparing the frequency of B cells to a reference value associated with the autoantibody-mediated disease in the subject, wherein the subject is in remission from the autoantibody-mediated disease if the frequency of B cells in the sample is lower than or equal to the reference value.

In an embodiment, the corticosteroid dose regimen is tapered to a lower dose amount or a lower dosing frequency. In an embodiment, the method further comprises administering to the subject a therapeutically effective amount of the second FcRn antagonist if the frequency of B cells in the sample is greater than or equal to the reference value. In an embodiment, the method further comprises administering to the subject a therapeutically effective amount of a second FcRn antagonist if the frequency of B cells in the sample is greater than or equal to the reference value.

In an embodiment, the reference value is about 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold or 7-fold more than a normal frequency of B cells.

In an embodiment, the normal frequency of B cells is about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of lymphocytes. In an embodiment, the reference value is about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of lymphocytes.

In an embodiment, the reference value is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the maximum frequency of B cells measured in the subject prior to receiving any treatment for the autoantibody-mediated disease. In an embodiment, the reference value is greater than about 60% of the maximum frequency of B cells measured in the subject prior to receiving any treatment for the autoantibody-mediated disease.

In an embodiment, the reference value is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the maximum frequency of B cells measured in the subject prior to receiving any treatment for the autoantibody-mediated disease. In an embodiment, the reference value is greater than 60% of the maximum frequency of B cells measured in the subject prior to receiving any treatment for the autoantibody-mediated disease.

In an embodiment, the reference value is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the maximum frequency of B cells measured in the subject prior to receiving any treatment for the autoantibody-mediated disease. In an embodiment, the reference value is greater than 65% of the maximum frequency of B cells measured in the subject prior to receiving any treatment for the autoantibody-mediated disease.

In an embodiment, the reference value is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% higher than the lowest frequency of B cells measured in the subject following treatment with the first FcRn antagonist for the autoantibody-mediated disease.

In an embodiment, the reference value is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% higher than the lowest frequency of B cells measured in the subject following treatment with the first FcRn antagonist for the autoantibody-mediated disease. In an embodiment, the reference value is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% higher than the lowest frequency of B cells measured in the subject following treatment with the first FcRn antagonist for the autoantibody-mediated disease.

In an embodiment, the reference value is about 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold or 7-fold more than the lowest frequency of B cells measured in the subject following treatment with the first FcRn antagonist for the autoantibody-mediated disease.

In an embodiment, the reference value is 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold or 7-fold more than the lowest frequency of B cells measured in the subject following treatment with the first FcRn antagonist for the autoantibody-mediated disease.

In an embodiment, the subject was previously treated with the first FcRn antagonist at a dose of about 10 mg/kg to about 30 mg/kg, administered intravenously. In an embodiment, the subject was previously treated with the first FcRn antagonist at a dose of about 10 mg/kg, administered intravenously. In an embodiment, the subject was previously treated with the first FcRn antagonist at a dose of about 15 mg/kg, administered intravenously. In an embodiment, the subject was previously treated with the first FcRn antagonist at a dose of about 20 mg/kg, administered intravenously. In an embodiment, the subject was previously treated with the first FcRn antagonist at a dose of about 25 mg/kg, administered intravenously. In an embodiment, the subject was previously treated with the first FcRn antagonist at a dose of about 30 mg/kg, administered intravenously.

In an embodiment, the subject was previously treated with the first FcRn antagonist at a dose of about 10 mg/kg to about 30 mg/kg once weekly, administered intravenously. In an embodiment, the subject was previously treated with the first FcRn antagonist at a dose of about 10 mg/kg once weekly, administered intravenously. In an embodiment, the subject was previously treated with the first FcRn antagonist at a dose of about 15 mg/kg once weekly, administered intravenously. In an embodiment, the subject was previously treated with the first FcRn antagonist at a dose of about 20 mg/kg once weekly, administered intravenously. In an embodiment, the subject was previously treated with the first FcRn antagonist at a dose of about 25 mg/kg once weekly, administered intravenously. In an embodiment, the subject was previously treated with the first FcRn antagonist at a dose of about 30 mg/kg once weekly, administered intravenously.

In an embodiment, the subject was previously treated with the first FcRn antagonist at a dose of about 10 mg/kg to about 30 mg/kg once every two weeks, administered intravenously. In an embodiment, the subject was previously treated with the first FcRn antagonist at a dose of about 10 mg/kg once every two weeks, administered intravenously. In an embodiment, the subject was previously treated with the first FcRn antagonist at a dose of about 15 mg/kg once every two weeks, administered intravenously. In an embodiment, the subject was previously treated with the first FcRn antagonist at a dose of about 20 mg/kg once every two weeks, administered intravenously. In an embodiment, the subject was previously treated with the first FcRn antagonist at a dose of about 25 mg/kg once every two weeks, administered intravenously. In an embodiment, the subject was previously treated with the first FcRn antagonist at a dose of about 30 mg/kg once every two weeks, administered intravenously.

In an embodiment, the subject was previously treated with the first FcRn antagonist at a dose of about 750 mg to about 3000 mg, administered subcutaneously. In an embodiment, the subject was previously treated with the first FcRn antagonist at a dose of about 750 mg, administered subcutaneously. In an embodiment, the subject was previously treated with the first FcRn antagonist at a dose of about 1000 mg, administered subcutaneously. In an embodiment, the subject was previously treated with the first FcRn antagonist at a dose of about 1250 mg, administered subcutaneously. In an embodiment, the subject was previously treated with the first FcRn antagonist at a dose of about 1500 mg, administered subcutaneously. In an embodiment, the subject was previously treated with the first FcRn antagonist at a dose of about 1750 mg, administered subcutaneously.

In an embodiment, the subject was previously treated with the first FcRn antagonist at a dose of about 750 mg to about 3000 mg once weekly, administered subcutaneously. In an embodiment, the subject was previously treated with the first FcRn antagonist at a dose of about 750 mg once weekly, administered subcutaneously. In an embodiment, the subject was previously treated with the first FcRn antagonist at a dose of about 1000 mg once weekly, administered subcutaneously. In an embodiment, the subject was previously treated with the first FcRn antagonist at a dose of about 1250 mg once weekly, administered subcutaneously. In an embodiment, the subject was previously treated with the first FcRn antagonist at a dose of about 1500 mg once weekly, administered subcutaneously. In an embodiment, the subject was previously treated with the first FcRn antagonist at a dose of about 1750 mg once weekly, administered subcutaneously.

In an embodiment, the subject was previously treated with the first FcRn antagonist at a dose of about 750 mg to about 3000 mg once every two weeks, administered subcutaneously. In an embodiment, the subject was previously treated with the first FcRn antagonist at a dose of about 750 mg once every two weeks, administered subcutaneously. In an embodiment, the subject was previously treated with the first FcRn antagonist at a dose of about 1000 mg once every two weeks, administered subcutaneously. In an embodiment, the subject was previously treated with the first FcRn antagonist at a dose of about 1250 mg once every two weeks, administered subcutaneously. In an embodiment, the subject was previously treated with the first FcRn antagonist at a dose of about 1500 mg once every two weeks, administered subcutaneously. In an embodiment, the subject was previously treated with the first FcRn antagonist at a dose of about 1750 mg once every two weeks, administered subcutaneously.

In an embodiment, the subject was also previously treated with a corticosteroid or an immunosuppressive agent. In an embodiment, the subject was previously treated with prednisone.

In an embodiment, the subject was previously treated with prednisone at a dose of <5 mg/kg/day. In an embodiment, the subject was previously treated with prednisone at a dose of <3 mg/kg/day. In an embodiment, the subject was previously treated with prednisone at a dose of <2 mg/kg/day. In an embodiment, the subject was previously treated with prednisone at a dose of <1 mg/kg/day. In an embodiment, the subject was previously treated with prednisone at a dose of <0.5 mg/kg/day. In an embodiment, the subject was previously treated with prednisone at a dose of <0.4 mg/kg/day. In an embodiment, the subject was previously treated with prednisone at a dose of <0.3 mg/kg/day. In an embodiment, the subject was previously treated with prednisone at a dose of <0.2 mg/kg/day. In an embodiment, the subject was previously treated with prednisone at a dose of <0.1 mg/kg/day.

In an embodiment, the effective amount of the second FcRn antagonist is a higher dose than the previous treatment with the first FcRn antagonist. In an embodiment, the effective amount of the second FcRn antagonist is a lower dose than the previous treatment with the first FcRn antagonist.

In an embodiment, the effective amount of the second FcRn antagonist is administered more frequently compared to the previous treatment with the first FcRn antagonist. In an embodiment, the effective amount of the second FcRn antagonist is administered less frequently compared to the previous treatment with the first FcRn antagonist.

In an embodiment, the effective amount of the second FcRn antagonist is administered intravenously at a dose of about 10 mg/kg to about 30 mg/kg once weekly, every two weeks, every three weeks, every four weeks, or every six weeks. In an embodiment, the effective amount of the second FcRn antagonist is administered intravenously at a dose of 10 mg/kg once weekly, every two weeks, every three weeks, every four weeks, or every six weeks. In an embodiment, the effective amount of the second FcRn antagonist is administered intravenously at a dose of 15 mg/kg once weekly, every two weeks, every three weeks, every four weeks, or every six weeks. In an embodiment, the effective amount of the second FcRn antagonist is administered intravenously at a dose of 20 mg/kg once weekly, every two weeks, every three weeks, every four weeks, or every six weeks. In an embodiment, the effective amount of the second FcRn antagonist is administered intravenously at a dose of 25 mg/kg once weekly, every two weeks, every three weeks, every four weeks, or every six weeks. In an embodiment, the effective amount of the second FcRn antagonist is administered intravenously at a dose of 30 mg/kg once weekly, every two weeks, every three weeks, every four weeks, or every six weeks.

In an embodiment, the effective amount of the second FcRn antagonist is administered intravenously at a dose of about 10 mg/kg once weekly, every two weeks, every three weeks, every four weeks, or every six weeks. In an embodiment, the effective amount of the second FcRn antagonist is administered intravenously at a dose of about 15 mg/kg once weekly, every two weeks, every three weeks, every four weeks, or every six weeks. In an embodiment, the effective amount of the second FcRn antagonist is administered intravenously at a dose of about 20 mg/kg once weekly, every two weeks, every three weeks, every four weeks, or every six weeks. In an embodiment, the effective amount of the second FcRn antagonist is administered intravenously at a dose of about 25 mg/kg once weekly, every two weeks, every three weeks, every four weeks, or every six weeks. In an embodiment, the effective amount of the second FcRn antagonist is administered intravenously at a dose of about 30 mg/kg once weekly, every two weeks, every three weeks, every four weeks, or every six weeks.

In an embodiment, the effective amount of the second FcRn antagonist is administered subcutaneously at a fixed dose of about 750 mg to about 3000 mg once weekly, every two weeks, every three weeks, every four weeks, or every six weeks. In an embodiment, the effective amount of the FcRn second antagonist is administered subcutaneously at a fixed dose of 1000 mg or 2000 mg once weekly, every two weeks, every three weeks, every four weeks, or every six weeks. In an embodiment, the effective amount of the second FcRn antagonist is administered subcutaneously at a fixed dose of about 750 mg once weekly, every two weeks, every three weeks, every four weeks, or every six weeks. In an embodiment, the effective amount of the second FcRn antagonist is administered subcutaneously at a fixed dose of about 1000 mg once weekly, every two weeks, every three weeks, every four weeks, or every six weeks. In an embodiment, the effective amount of the second FcRn antagonist is administered subcutaneously at a fixed dose of about 1250 mg once weekly, every two weeks, every three weeks, every four weeks, or every six weeks. In an embodiment, the effective amount of the second FcRn antagonist is administered subcutaneously at a fixed dose of about 1500 mg once weekly, every two weeks, every three weeks, every four weeks, or every six weeks. In an embodiment, the effective amount of the second FcRn antagonist is administered subcutaneously at a fixed dose of about 1750 mg once weekly, every two weeks, every three weeks, every four weeks, or every six weeks.

In an embodiment, the effective amount of the second FcRn antagonist is administered subcutaneously at a fixed dose of 750 mg once weekly, every two weeks, every three weeks, every four weeks, or every six weeks. In an embodiment, the effective amount of the second FcRn antagonist is administered subcutaneously at a fixed dose of 1000 mg once weekly, every two weeks, every three weeks, every four weeks, or every six weeks. In an embodiment, the effective amount of the second FcRn antagonist is administered subcutaneously at a fixed dose of 1250 mg once weekly, every two weeks, every three weeks, every four weeks, or every six weeks. In an embodiment, the effective amount of the second FcRn antagonist is administered subcutaneously at a fixed dose of 1500 mg once weekly, every two weeks, every three weeks, every four weeks, or every six weeks. In an embodiment, the effective amount of the second FcRn antagonist is administered subcutaneously at a fixed dose of 1750 mg once weekly, every two weeks, every three weeks, every four weeks, or every six weeks.

In an embodiment, the method further comprises administering to the subject an effective amount of a corticosteroid or an immunosuppressive agent. In an embodiment, the effective amount of the corticosteroid is administered at a dose of about 0.5 mg/kg per day. In an embodiment, the effective amount of the corticosteroid is administered at a dose of about 0.25 mg/kg per day. In an embodiment, the effective amount of the corticosteroid is administered at a dose of about 20 mg per day. In an embodiment, the effective amount of the corticosteroid is administered at a dose of about 10 mg per day. In an embodiment, the effective amount of the corticosteroid is administered at a dose of about 5 mg/kg/day. In an embodiment, the effective amount of the corticosteroid is administered at a dose of about 3 mg/kg/day. In an embodiment, the effective amount of the corticosteroid is administered at a dose of about 2 mg/kg/day. In an embodiment, the effective amount of the corticosteroid is administered at a dose of about 1 mg/kg/day. In an embodiment, the effective amount of the corticosteroid is administered at a dose of about 0.5 mg/kg/day. In an embodiment, the effective amount of the corticosteroid is administered at a dose of about 0.4 mg/kg/day. In an embodiment, the effective amount of the corticosteroid is administered at a dose of about 0.3 mg/kg/day. In an embodiment, the effective amount of the corticosteroid is administered at a dose of about 0.2 mg/kg/day. In an embodiment, the effective amount of the corticosteroid is administered at a dose of about 0.1 mg/kg/day.

In an embodiment, the subject the corticosteroid dosing regimen is tapered based on the amount of B cells in a subject with an autoantibody-mediated disease. In an embodiment, tapering the corticosteroid regimen is lowering the dose or lowering the dosing frequency of the corticosteroid. In an embodiment, the tapered corticosteroid dose is <2 mg prednisone/kg/day or equivalent. In an embodiment, the tapered corticosteroid dose is less than or equal to about 1.5, 1.0, 0.75, 0.5, or 0.2 mg prednisone/kg/day or equivalent. In an embodiment, the tapered corticosteroid dose is <0.5 mg prednisone/kg/day or equivalent.

In an embodiment, the frequency of B cells is measured with flow cytometry. In an embodiment, the B cells are CD19+ B cells.

In an embodiment, the first FcRn antagonist and the second FcRn antagonist are each the same FcRn antagonist. In an embodiment, the first FcRn antagonist and the second FcRn antagonist are each a different FcRn antagonist.

In an embodiment, the FcRn antagonist is an anti-FcRn antibody. In an embodiment, the first FcRn antagonist is an anti-FcRn antibody. In an embodiment, the second FcRn antagonist is an anti-FcRn antibody.

In an embodiment, the anti-FcRn antibody is rozanolixizumab (UCB7665), nipocalimab (M281), orilanolimab (ALXN1830/SYNT001), or batoclimab (IMVT-1401/RVT1401/HBM9161).

In an embodiment, the FcRn antagonist is an Fc region comprising amino acids Y, T, E, K, F, and Y at EU positions 252, 254, 256, 433, 434, and 436, respectively. In an embodiment, the first FcRn antagonist or the second FcRn antagonist is an Fc region comprising amino acids Y, T, E, K, F, and Y at EU positions 252, 254, 256, 433, 434, and 436, respectively.

In an embodiment, the FcRn antagonist is efgartigimod. In an embodiment, the first FcRn antagonist or the second FcRn antagonist is efgartigimod.

In an embodiment, the FcRn antagonist comprises the amino acid sequence of SEQ ID NO: 1, 2, or 3. In an embodiment, the first FcRn antagonist or the second FcRn antagonist comprises the amino acid sequence of SEQ ID NO: 1, 2, or 3

In an embodiment, the first FcRn antagonist is an anti-FcRn antibody and the second FcRn antagonist is efgartigimod. In an embodiment, the first FcRn antagonist is an anti-FcRn antibody and the second FcRn antagonist comprises the amino acid sequence of SEQ ID NO: 1, 2, or 3. In an embodiment, the anti-FcRn antibody is rozanolixizumab (UCB7665), nipocalimab (M281), orilanolimab (ALXN1830/SYNT001), or batoclimab (IMVT-1401/RVT1401/HBM9161). In an embodiment, the patient has not been previously treated with efgartigimod.

In an embodiment, the first FcRn antagonist is selected from the group consisting of rozanolixizumab (UCB7665), nipocalimab (M281), orilanolimab (ALXN1830/SYNT001), or batoclimab (IMVT-1401/RVT1401/HBM9161) and the second FcRn antagonist is an Fc region comprising amino acids Y, T, E, K, F, and Y at EU positions 252, 254, 256, 433, 434, and 436, respectively. In an embodiment, the first FcRn antagonist is selected from the group consisting of rozanolixizumab (UCB7665), nipocalimab (M281), orilanolimab (ALXN1830/SYNT001), or batoclimab (IMVT-1401/RVT1401/HBM9161) and the second FcRn antagonist is efgartigimod.

In an embodiment, the subject has a serum level of a pathogenic IgG autoantibody that is associated with a relapse of the autoantibody-mediated disease. In an embodiment, the pathogenic IgG autoantibody is an anti-Dsg-3 antibody or an anti-Dsg-1 antibody. In an embodiment, the level of a pathogenic IgG autoantibody is measured by ELISA. In an embodiment, the serum level of a pathogenic IgG autoantibody is compared to baseline levels in the subject.

In an embodiment, the autoantibody-mediated disease is selected from the group consisting of: allogenic islet graft rejection, alopecia areata, ankylosing spondylitis, antiphospholipid syndrome (APS), autoimmune Addison's disease, Alzheimer's disease, antibody-mediated allograft rejection (AMR), antineutrophil cytoplasmic autoantibodies (ANCA), ANCA vasculitis, autoimmune diseases of the adrenal gland, autoimmune encephalitis, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenia purpura or idiopathic thrombocytopenia purpura or immune-mediated thrombocytopenia), autoimmune urticaria, Behcet's disease, bullous pemphigoid (BP), cardiomyopathy, Castleman's syndrome, celiac spruce-dermatitis, chronic fatigue immune disfunction syndrome, chronic inflammatory demyelinating polyneuropathy (CIDP), Churg-Strauss syndrome, cicatricial pemphigoid, CREST syndrome, cold agglutinin disease, COVID-19 mediated postural orthostatic tachycardia syndrome (POTS), Crohn's disease, delayed graft function after kidney transplant, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre syndrome (GBS), Goodpasture's syndrome, graft-versus-host disease (GVHD), Hashimoto's thyroiditis, hemophilia A, hemolytic disease of the fetus and newborn (HDFN), idiopathic membranous neuropathy, idiopathic pulmonary fibrosis, IgA neuropathy, IgM polyneuropathies, juvenile arthritis, Kawasaki's disease, lichen planus, lichen sclerosus, lupus erythematosus, lupus nephritis, membranous neuropathy, membranous nephropathy, Ménière's disease, mixed connective tissue disease, mucous membrane pemphigoid, multiple sclerosis, Type 1 diabetes mellitus, multifocal motor neuropathy (MMN), myelin oligodendrocyte glycoprotein antibody-associated disease (MOGAD), myasthenia gravis (MG), generalized myasthenia gravis (gMG), ocular myasthenia gravis (OMG), myositis, neuromyelitis optica (NMO), paraneoplastic bullous pemphigoid, pemphigoid gestationis, pemphigus vulgaris (PV), pemphigus foliaceus (PF), pernicious anemia, polyarteritis nodosa, polychrondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Raynaud's phenomenon, Reiter's syndrome, rheumatoid arthritis (RA), sarcoidosis, scleroderma, Sjögren's syndrome, solid organ transplant rejection, stiff-man syndrome, systemic lupus erythematosus (SLE), Takayasu's arteritis, toxic epidermal necrolysis (TEN), Stevens-Johnson syndrome (SJS), temporal arteritis/giant cell arteritis, thrombotic thrombocytopenia purpura (TTP), thyroid eye disease, ulcerative colitis, uveitis, warm autoimmune hemolytic anemia (wAIHA), dermatitis herpetiformis vasculitis, anti-neutrophil cytoplasmic antibody-associated vasculitides, vitiligo, and Wegner's granulomatosis.

In an embodiment, the autoantibody-mediated disease is pemphigus vulgaris (PV). In an embodiment, the autoantibody-mediated disease is pemphigus foliaceus (PF).

In an embodiment, the subject has one or more physical symptoms of an autoantibody-mediated disease following treatment with the first FcRn antagonist. In an embodiment, the one or more physical symptoms include but are not limited to ocular muscle fatigue or weakness, skeletal muscle fatigue or weakness, respiratory muscle fatigue or weakness, disabling fatigue, slurred speech, choking, impaired swallowing, double or blurred vision, immobility requiring assistance, shortness of breath, respiratory failure, and blisters, including skin and mouth blisters.

### EXAMPLES

The following examples are offered by way of illustration, and not by way of limitation.

### Example 1: Analysis of Clinical, Serological, and Immunological Parameters in Pemphigus, a Model IgG-Mediated Organ Specific Autoimmune Disease, Following Treatment with Efgartigimod

To investigate changes in the systemic immune signatures during extended anti-FcRn treatment, autoreactive and protective anti-infective antibody titers, and B cell and T cell phenotypes were assessed in peripheral blood of pemphigus patients treated with efgartigimod up to 34 weeks.

### A. METHODS

### Study Design and Treatment Intervention

Extended treatment with efgartigimod was gradually introduced in an open-label, non-controlled phase 2 trial (NCT03334058) in patients with mild to moderate Pemphigus vulgaris (PV) or pemphigus foliaceus (PF). Seven patients (7 PV) were treated for 15 weeks in cohort 3 and 15 patients (8 PV, 7 PF) were treated up to 34 weeks in cohort 4. PV or diagnosis was confirmed by positive direct immunofluorescence and positive indirect immunofluorescence, and/or desmoglein (Dsg)-1/3 ELISA. Patients were either newly diagnosed, or relapsing, with mild to moderate disease severity (pemphigus disease area index (PDAI)<45 at baseline). The treatment period was preceded by a screening period up to 3 weeks and followed by a treatment-free, follow-up period of 10 weeks. In cohort 3, intravenous (IV) efgartigimod was administered weekly at 10 mg/kg for 4 weeks as induction period followed by IV administration every other week for 12 weeks as a maintenance period. In cohort 3, efgartigimod was initiated as monotherapy or in combination with 20 mg/day of prednisone at the discretion of the investigator, and prednisone was tapered starting in the maintenance period. In cohort 4, IV efgartigimod was dosed weekly, at 25 mg/kg body weight until the end of consolidation (EoC, defined as the time at which no new lesions have developed for a minimum of 2 weeks and the majority, i.e., approximately 80% of established lesions have healed) after which, patients were dosed with efgartigimod at 25 mg/kg every other week.

In cohort 4, efgartigimod was initiated in association with concomitant prednisone (20 mg/day) in all newly diagnosed patients and relapsing patients off therapy, or at the tapered dose at which relapse occurred. The oral prednisone dose could be tapered as of EoC. No other systemic treatments for pemphigus were permitted during the study, whereas topical corticosteroids, analgesics, and supportive care for corticosteroid therapy (e.g., vitamin D, proton-pump inhibitors, specific diets) were allowed.

### Ethics

The study was conducted in accordance with the Good Clinical Practice guidelines, in conformity with the ethical principles of the Declaration of Helsinki and was compliant with all relevant country-specific laws. The study protocol and all other appropriate study-related information were reviewed and approved by the ethics committees or institutional review boards of every center.

### Immunoglobulin Assays

Pharmacodynamic analysis included serum levels of total IgG, IgG subclasses, and anti-Dsg-1 and anti-Dsg-3 autoantibodies by ELISA (Euroimmun, Germany). Serum levels of protective vaccine antibodies against tetanus toxoid (TT, Indirect EIA, Virotech), varicella zoster virus (VZV, CLIA, Diasorin) and pneumococcal capsular polysaccharide (PCP, EIA, The Binding Site Group) were measured for all patients. A customized Addressable Laser Bead ImmunoAssay (ALBIA) test was performed to determine the anti-Dsg-3 IgG subclasses at different timepoints. Briefly, 20 μg of anti-HIS antibody (clone HIS.H8) was coupled to 1.25×10⁶ fluorescent Bio-Plex R COOH-microspheres (Bio-Rad, USA) with the Bio-PlexR amine coupling kit (Bio-Rad) according to manufacturer's protocol. After coupling, coated beads were either used immediately or stored at −20° C. in the dark. Before use, 100 ng of recombinant Dsg-3 expressing a 6×His Tag were incubated with 1000 beads for 15 min at room temperature (RT) and then washed. Immediately prior to their use, coated beads were vigorously agitated for 30 seconds. Then, 10 μL of beads (containing 1000 beads) was added to 100 μL of patients or controls serum diluted 1/100 in PBS with Ca²⁺ and Mg²⁺ supplemented with 1% FCS; in Bio-Plex Pro Flat bottom plates (Bio-Rad). Plates were incubated for 90 minutes at RT in the dark on a plate shaker at 850 rpm. Blank (no serum), negative controls (healthy donor serum), and positive controls (human anti-Dsg-3 positive serum) were included in every assay. Beads were collected with a magnetic washer (Bio-Rad) and washed three times with 150 μL PBS 1× containing 0.1% Tween-20. Biotinylated mouse anti-human IgG-subclass specific secondary antibody (Southern Biotech, USA) was added (at 1/125 dilution for anti-IgG1 and for anti-IgG2, at 1/200 dilution for anti-IgG3 and for anti-IgG4), for 45 minutes at RT under shaking conditions. After washing, beads were incubated with 50 μL of streptavidin-R-phycoerythrin at 1/400 dilution for 15 min. Finally, beads were resuspended in 100 μL of PBS and mean fluorescence intensity (MFI) was determined on a Bio-PlexR apparatus using the Bio-PlexR Manager Software 4.0 (Bio-Rad). For determination of Dsg-3-reactive antibodies, the positivity threshold was set at the mean value obtained from 36 healthy donors plus 2 standard deviations. Anti-Dsg-1 ELISA (Euroimmun, Germany), with modifications to the detection system, was employed to identify anti-Dsg-1 IgG subclasses. Specifically, in the ELISA, the rabbit anti-human IgG HRP detection antibody was replaced by anti-human IgG1 HRP or anti-human IgG4 HRP (Southern Biotech, USA) to detect Dsg-1 specific IgG subclasses and used at 1/10,000 dilution. CIC-C1q EIA kit (A001, Quidel) was employed to detect levels of C1q-associated IgG aggregates (circulating immune complexes (CIC)) in sera of selected patients at different timepoints according to manufacturer's protocol.

### Isolation of Peripheral Blood Mononuclear Cells (PBMC)

A total of 60-80 mL of whole blood was collected into ten BD Vacutainer CPT tubes, mixed immediately, and centrifuged within two hours of blood collection at room temperature (RT) to separate PBMC and red blood cells. Following centrifugation, PBMC were resuspended into the plasma and transported to the analytical laboratory at Philipps University Marburg within 24 hours. Upon sample receipt, tubes were subjected to PBMC purification and flow cytometry analysis. The remaining cells were cryopreserved for later analysis.

### Phenotypic Profiling by Multiparameter Flow Cytometry and Detection of Dsg3-Specific B Cells

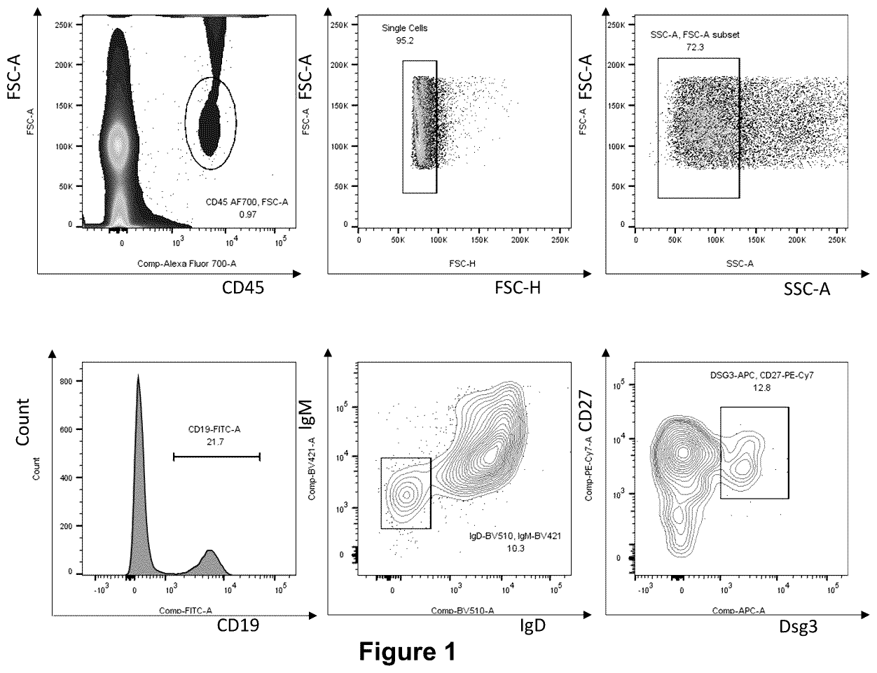

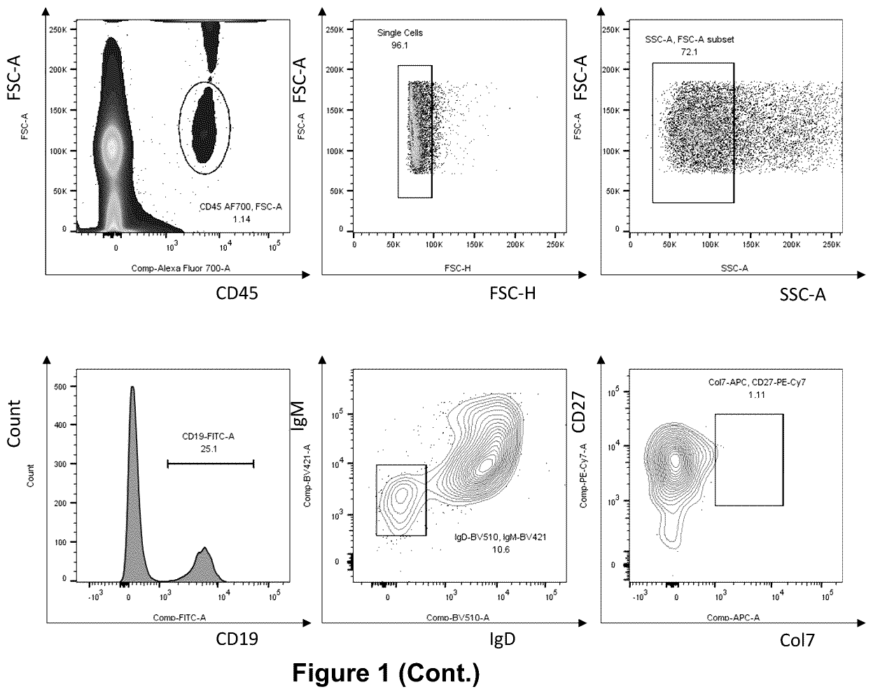

PBMCs were washed twice with PBS+1% FCS and 1×10⁶ cells were stained for T cell and B cell analysis including detection of Dsg3-specific B cells. The following antibodies were used for T cell panel analysis: mouse anti-human CD4 (RPA-T4, Biolegend), mouse anti-human CD45RA (HI100, Biolegend), mouse anti-human CXCR5 (J252D4, Biolegend), mouse anti-human CD25 (M-A251, Biolegend), mouse anti-human CD127 (A019D5, Biolegend), mouse anti-human CXCR3 (G025H7, Biolegend), and mouse anti-human CCR6 (G034E3, Biolegend). The following antibodies were used for B cell panel analysis: mouse anti-human CD45 (2D1, Biolegend), mouse anti-human CD19 (HIB19, Biolegend), mouse anti-human CD27 (M-T271, Biolegend), mouse anti-human CD38 (HB-7, Biolegend), mouse anti-human CD24 (ML5, Biolegend), mouse anti-human IgM (MHM-88, Biolegend), mouse anti-human IgD (IA6-2, Biolegend), and mouse anti-human CD138 (MI15, Biolegend). AlexaFluor 647 labeled recombinant human Dsg3 (extracellular domain, aa 1-566), produced in a baculovirus expression system, was included in a separate B cell staining panel. The gating strategy is shown in FIG. 1 for one representative patient.

### Dsg-Specific B Cell Detection Using ELISPOT Assay

The frequencies of circulating total IgG and Dsg-specific IgG-antibody secreting cells (ASC) were determined by human IgG ELISPOT Basic assays (Mabtech, Nacka Strand, Sweden). PBMCs from pemphigus patients from the clinical trial described above were pre-stimulated with R848 (1 μg/mL) and rhIL2 (10 ng/mL) in complete medium (RPMI-1640 supplemented with 10% fetal bovine serum, 2 mM L-glutamine, 100 U/mL penicillin, and 100 μg/mL streptomycin) in 96-well plates for 72 hours at 37° C. Plates of ELISPOT MAIPS-4510 (Merck Millipore, Darmstadt, Germany) were coated overnight at 4° C. with anti-IgG human Abs. The plates were washed and blocked with complete medium before use. Pre-stimulated PBMCs were washed, resuspended in complete medium, and transferred to the plate, and then incubated for 24 hours with 1×10⁵-4×10⁵ PBMCs per well to detect anti-Dsg-1 and anti-Dsg-3 IgG-secreting ASC, and with 2.5×10³-1×10⁴ PBMCs per well to detect total IgG-ASC. IgG-ASC were detected by addition of biotinylated mouse IgG anti-human IgG. Frequencies of anti-Dsg-1 or anti-Dsg-3 IgG secreting ASC were calculated after incubation for 2 hours with histidine-tagged recombinant Dsg-1 or Dsg-3 proteins (1 μg/mL) in phosphate-buffered saline with calcium (Eurobio, Les Ulis, France). Biotinylated anti-histidine antibodies (0.5 μg/mL) (Abcam, Cambridge, United Kingdom) were then added. The streptavidin conjugated peroxidase and substrate tetramethylbenzidine were used to detect spots. The number of spots were determined with ELISPOT Plate Readers and ImmunoSpot software (CTL Europe GmbH, Bonn, Germany). Results were expressed as frequencies of Dsg-specific IgG-ASC among total IgG-ASC. The sensitivity and specificity of the ELISPOT assay were estimated at 1 Dsg-3 specific ASC/105 total ASC, and 100%, respectively.

### Statistical Analyses

Descriptive statistical methods were used to analyze the data. Summaries (mean, standard error, median, range) were plotted graphically by study days/time points.

### B. RESULTS

### Sustained Clinical Response and Autoantibody Suppression

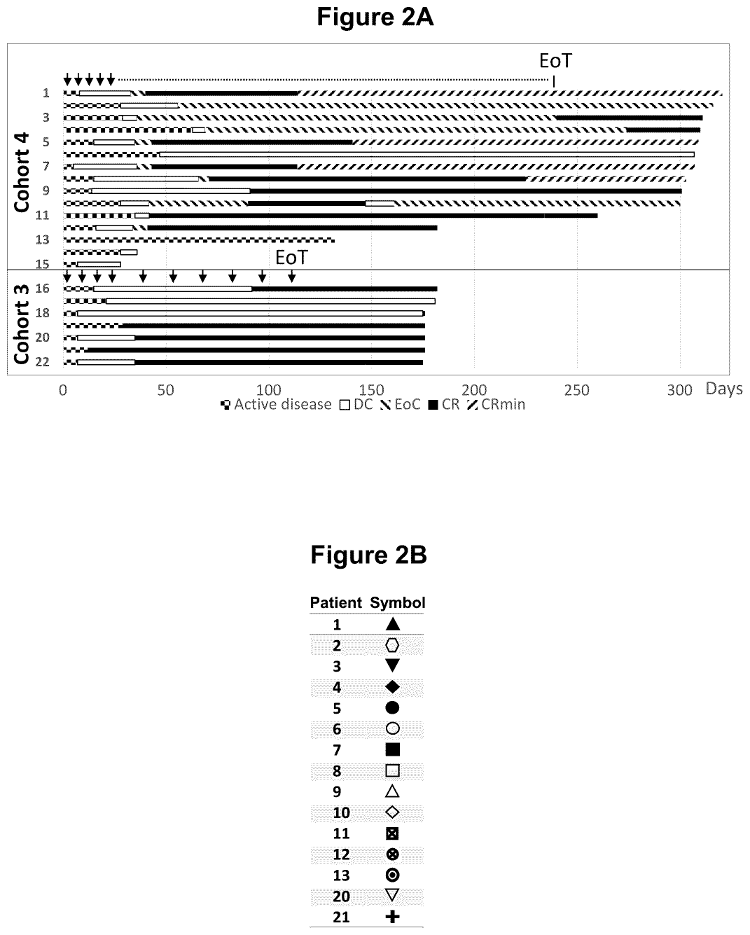

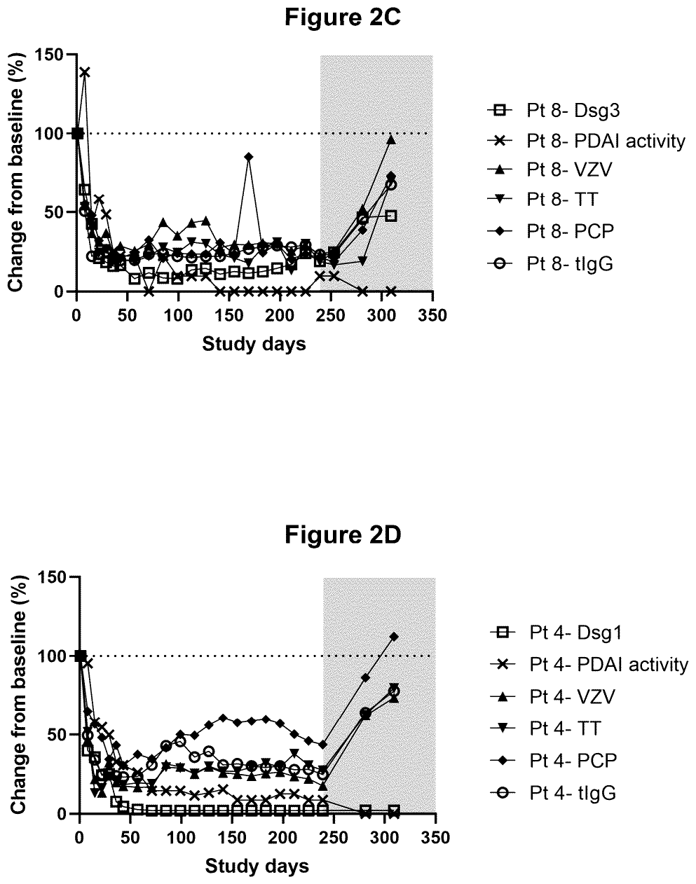

The clinical outcomes and characteristics of patients with PV and PF in cohorts 3 and 4, who received extended treatment of efgartigimod (15 and up to 34 weeks, respectively), are shown in Table 6, and the clinical outcomes are shown in FIG. 2A (symbols used throughout the Figures for selected patients are shown in FIG. 2B). Nine patients in cohort 4 (patients 1, 3, 4, 5, 7, 8, 9, 11, and 12) reached CR at any point during the trial while receiving efgartigimod (until day 239) and low-dose prednisone, of which six patients (patients 1, 4, 5, 7, 8, and 9) were in CR at the end of study (end of 10-week efgartigimod-free period). Four patients reached complete clinical remission on minimal therapy (CRmin), defined as the absence of new or established lesions while the patient is receiving minimal therapy where minimal therapy is defined as less than or equal to 10 mg/day of prednisone (or the equivalent) for at least two months, at any point during the study. Patient 3 exhibited low disease activity with a PDAI activity score of 3 following CR and two patients (11 and 12) relapsed to a PDAI activity score >10. Three patients (patients 2, 6, and 10) improved clinically and maintained clinical improvement status until end of study: patient 2 and 10 reached and maintained EoC, patient 6 reached DC and had a PDAI activity score of 1 at end of study. In cohort 3, five of seven patients achieved CR at any point during the study (patients 16, 19, 20, 21, and 22) and four of these patients were in CR at the end of treatment-free follow up (patients 16, 20, 21, and 22).

TABLE 6
Baseline characteristics of patients receiving extended
efgartigimod treatment in cohorts 3 and 4.

| | | | | | PDAI | |
|---|---|---|---|---|---|---|
| | Pemphigus | | | | activity | |
| | Disease | | | Type of | at | Sub |
| patient | History | Gender | Age | Pemphigus | baseline | analyses |
| | Cohort 4 | | | | | |
| 1 | Relapsing | F | 63 | PF | 9 | S, CI |
| 2 | Relapsing | F | 48 | PV M | 27.6 | S |
| 3 | Relapsing | M | 57 | PF | 20.3 | S, CI |
| 4 | Newly | F | 42 | PF | 34.5 | S, CI, P |
| | diagnosed | | | | | |
| 5 | Relapsing | F | 62 | PV M | 14.6 | S, CI |
| 6 | Relapsing | M | 66 | PV MC | 11 | S, CI |
| 7 | Relapsing | F | 85 | PF | 11.2 | S, CI |
| 8 | Newly | M | 47 | PV MC | 10.3 | S, CI, P |
| | diagnosed | | | | | |
| 9 | Newly | M | 67 | PF | 19 | S, CI |
| | diagnosed | | | | | |
| 10 | Relapsing | F | 36 | PF | 30.2 | S, CI |
| 11 | Newly | M | 58 | PV C | 2 | CI |
| | diagnosed | | | | | |
| 12 | Newly | F | 66 | PV MC | 7.3 | CI |
| | diagnosed | | | | | |
| 13 | Newly | F | 51 | PF | 30.3 | CI |
| | diagnosed | | | | | |
| 14 | Newly | M | 22 | PV MC | 39.9 | |
| | diagnosed | | | | | |
| 15 | Relapsing | M | 30 | PV C | 28.4 | |
| | Cohort 3 | | | | | |
| 16 | Newly | F | 40 | PV M | 2 | |
| | diagnosed | | | | | |
| 17 | Relapsing | M | 36 | PV MC | 3 | |
| 18 | Relapsing | F | 48 | PV M | 12 | |
| 19 | Relapsing | F | 52 | PV M | 1 | |
| 20 | Newly | F | 65 | PV MC | 23 | S |
| | Diagnosed | | | | | |
| 21 | Relapsing | F | 30 | PV MC | 18.9 | S |
| 22 | Relapsing | F | 54 | PV MC | 14 | |

List of patients, pemphigus disease history, gender, age, type of pemphigus, and PDAI activity at baseline. PDAI, pemphigus disease area index; PV, pemphigus vulgaris; PF, pemphigus foliaceus; M, mucosal-dominant; MC, mucocutaneous; C, cutaneous. The sub analyses column indicates the type of analysis performed: S, serology; CI, cellular immunity; P, photography.

FIGS. 2C and 2D show a representative PV patient and a representative PF patient, respectively, from cohort 4 who both responded to efgartigimod treatment. Their clinical response is demonstrated by improvement of obvious physical symptoms and by their clinical and serological profiles (FIG. 2C and FIG. 2D). The PV patient (patient 8) had a PDAI activity score of 10.3 at baseline, which showed a decline starting after the first treatment (FIG. 2C). Further, the PV patient's mucosal blisters were healed by day 15. The PF patient had a PDAI activity score of 34.5 at baseline, which also showed a decline following the first treatment. The skin lesions of the PF patient declined gradually over time and were resolved completely by day 281 (FIG. 2D). Importantly, both patients showed no disease activity during the 10-week observation period after last efgartigimod treatment.

In the representative patients, total IgG, and TT-, PCP-, and VZV-specific antibody levels remained suppressed to about 70% in the treatment period, which recovered to baseline levels during treatment-free follow up (FIG. 2C and FIG. 2D). An early and prolonged reduction of anti-Dsg antibody levels was observed in both patients. During the ten weeks of treatment-free follow-up, there was no return of anti-Dsg1 antibodies in the PF patient (patient 4) and there was a reappearance of anti-Dsg3 antibodies to lower levels than before treatment (roughly 50%) in the PV patient (patient 8) (FIG. 2C and FIG. 2D). Patient 4 received 0.15 mg/kg/day of prednisone during efgartigimod-free follow up and was further tapered to 0.11 mg/kg/day at the last study visit, while patient 8's dose was 0.16 mg/kg/day during the efgartigimod-free 10-week period.

### Suppression of Anti-Dsg Autoantibodies Subtypes

In pemphigus, the disease is mainly attributed to the formation of autoantibodies to desmoglein (Dsg), a group of transmembrane desmosomal glycoproteins responsible for structural integrity of the epidermis. For pemphigus vulgaris (PV) these primarily target Dsg-3 and in pemphigus foliaceus (PF) these antibodies primarily target Dsg-1.

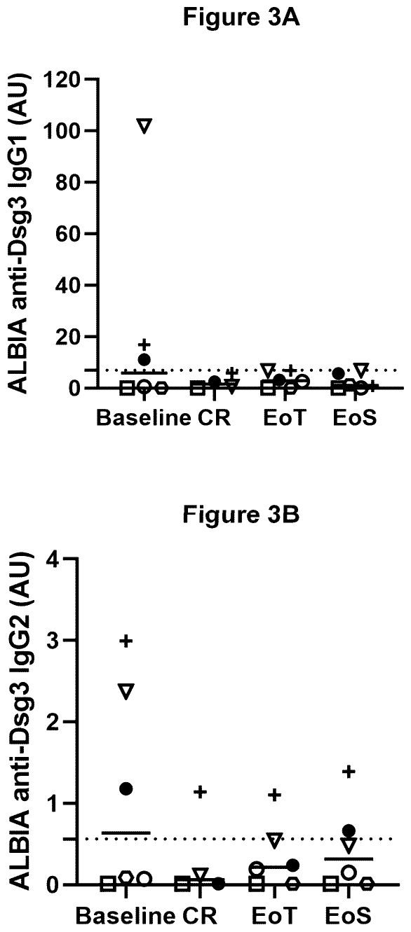

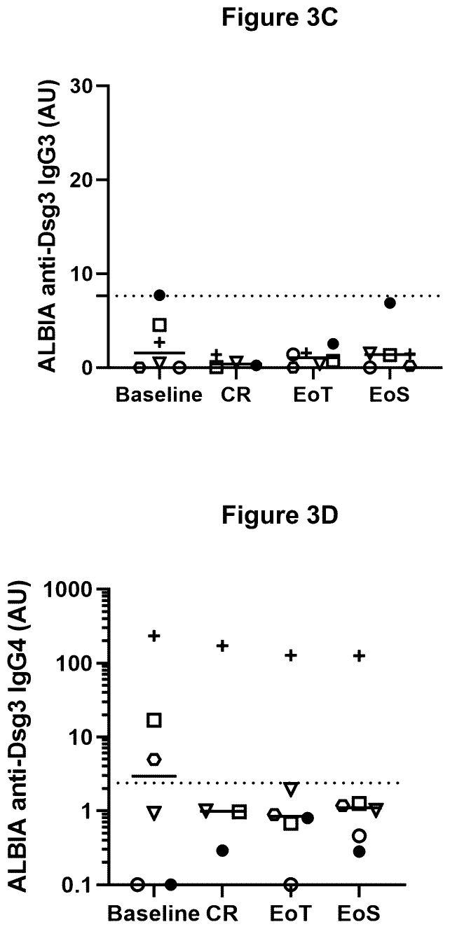

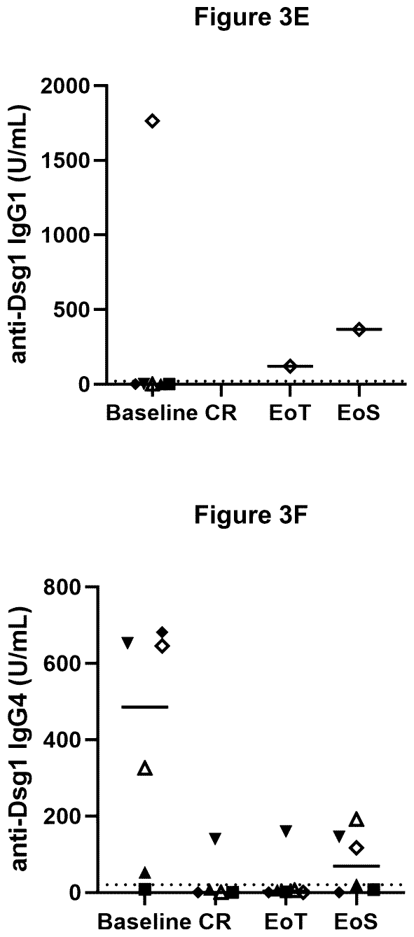

Because PV and PF primarily involve IgG4, and other IgG subtypes to a lesser extent, an ALBIA Dsg-3-assay was performed in six patients with P and a modified anti-Dsg-1 Euroimmun ELISA was performed in six patients with PF who received extended efgartigimod treatment in cohorts 3 and 4 and achieved a sustained clinical response. Patients used in these analyses are specified in Table 6 and anti-Dsg-1/3 titers are summarized in Table 7. At baseline, heterogenous anti-Dsg-1/3 IgG1-4 subclasses were detected in different patients. In patients with baseline levels above the positivity threshold, reduction of anti-Dsg-3 IgG1 autoantibodies to below the positivity threshold in 3 of 3 patients (FIG. 3A) and anti-Dsg-3 IgG4 in 2 of 3 patients (FIG. 3D) was observed. Anti-Dsg-3 IgG2 autoantibodies, present in 3 patients, were markedly reduced although only 1 patient decreased below the positivity threshold at end of study (FIG. 3B); IgG3 autoantibodies present in one patient at baseline became suppressed below the positivity threshold (FIG. 3C). The six patients with PF subjected to subclass analysis demonstrated a dominant IgG4 signature of anti-Dsg-1 autoantibodies (FIG. 3F), while one patient presented with IgG1 autoantibodies (FIG. 3E). Analysis of Dsg-1 IgG titers over time showed reduction of Dsg-1 specific IgG1 and IgG4 antibodies following clinical response which remained suppressed at the end of study (FIG. 3E and FIG. 3F). Thus, it can be concluded that efgartigimod decreases anti-Dsg titer regardless of the IgG subtype analyzed.

These results demonstrate that efgartigimod treatment leads to reduction of pemphigus disease causing autoantibodies and that the reduction is maintained for an extended time period after treatment. Further, clinical benefit in patients was directly linked to decreasing titers of pathogenic autoantibodies. Reduction of anti-Dsg-1 IgG was more consistent across patients, while change in anti-Dsg-3 IgG levels was more variable.

TABLE 7
Summary of individual clinical and serological responses by patients
from cohort 3 and 4 who achieved sustained clinical response.

| | | PDAI activity | | | Anti-Dsg-1 titer | | | Anti-Dsg-3 titer | | |
|---|---|---|---|---|---|---|---|---|---|---|
| Patient | Diagnosis | BL | EoT | EoS | BL | EoT | EoS | BL | EoT | EoS |
| 2 | PV | 27.6 | 3 | 2 | 79 | ND | ND | 380 | 168 | 316 |
| 5 | PV | 14.6 | 0 | 0 | 18 | ND | ND | 4440* | 504 | 632 |
| 6 | PV | 11 | 3 | 1 | — | — | — | 728 | 232 | 276 |
| 8 | PV | 10.3 | 1 | 0 | 69 | ND | ND | 920* | 152 | 380 |
| 20 | PV | 23 | 0 | 0 | 129 | ND | ND | 2208* | 63 | 272 |
| 21 | PV | 18.9 | 0 | 0 | 252 | 151 | 80 | 1738* | 452 | 412 |
| 1 | PF | 9 | 0 | 0 | 75 | 12 | 29 | — | — | — |
| 3 | PF | 20.3 | 2.9 | 3 | 252 | 40 | 76 | — | — | — |
| 4 | PF | 34.5 | 3 | 0 | 444 | ND | ND | — | — | — |
| 7 | PF | 11.2 | 0 | 0 | 62 | 29 | 29 | — | — | — |
| 9 | PF | 19 | 1 | 0 | 700 | 115 | 652 | 52 | ND | ND |
| 10 | PF | 30.2 | 5.3 | 6.3 | 660 | 112 | 127 | — | — | — |

BL, baseline; EoT, end of treatment; EoS, end of study; ND, nondetectable; PDAI, pemphigus disease area index; PF, pemphigus foliaceus; PV, pemphigus vulgaris.
*values determined in the research laboratory on leftover serum samples. During the trial 800 RU/mL was upper limit of quantification

### Suppression of Circulating Immune Complexes

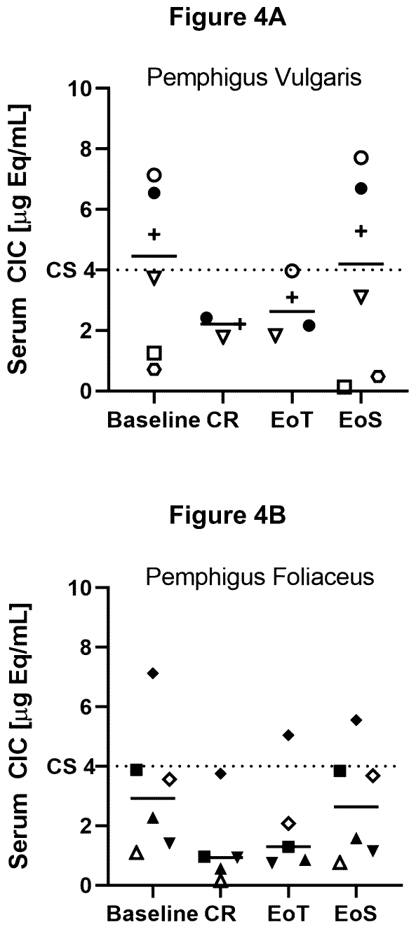

Immunocomplexes are found in healthy individuals, but their formation can be expected to be elevated in autoimmune diseases and partly drive and/or exaggerate their pathologies. As the half-life of immune complexes, largely containing IgG, may also be affected by the biology of FcRn, levels of CIC were investigated in the same six patients with PV and six patients with PF. These patients were subjected to IgG CIC analysis by C1q ELISA, which detects complement-fixed IgG antibodies. IgG CIC levels are considered clinically significant if ≥4.0 μg Eq/mL. Four patients presented elevated CIC levels, but notable reduction of CICs during treatment with efgartigimod was observed (FIG. 4A and FIG. 4B), consistent with the observed improvement in their clinical condition.

### Changes in Dsg-3+ and Dsg-1+ B Cells

Based on the observation of prolonged suppression of anti-Dsg autoantibodies compared to overall serum IgG in pemphigus patients following efgartigimod treatment, PBMCs of PV patients were analyzed to determine the fate of Dsg-3-specific B cells. PBMC collection was performed in cohort 4 patients only (n=15) and were successfully isolated in 12 patients (5 PV, 7 PF) of which 9 (3 PV, 6 PF) achieved a sustained clinical response.

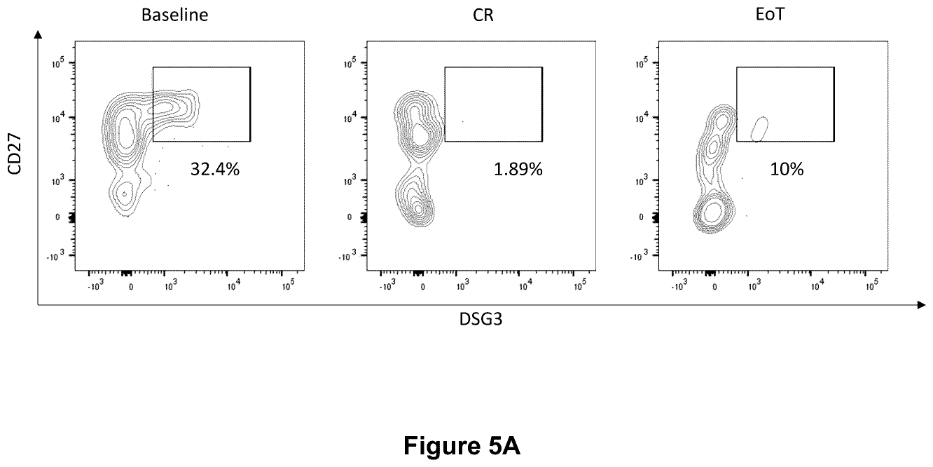

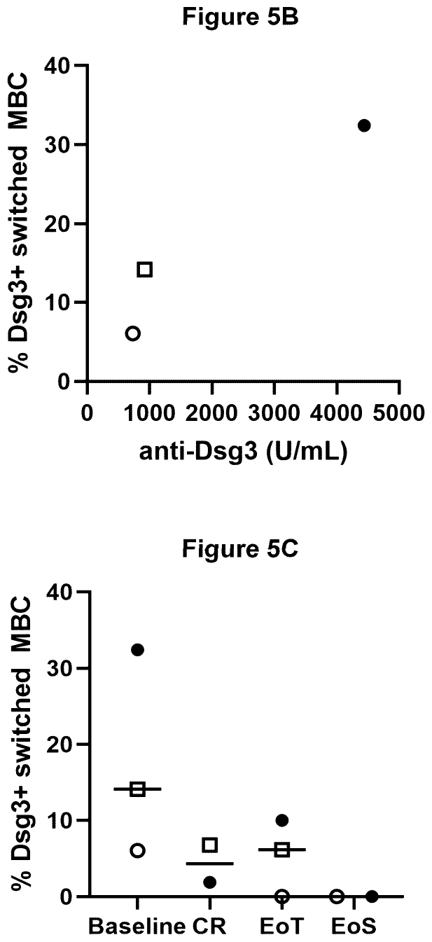

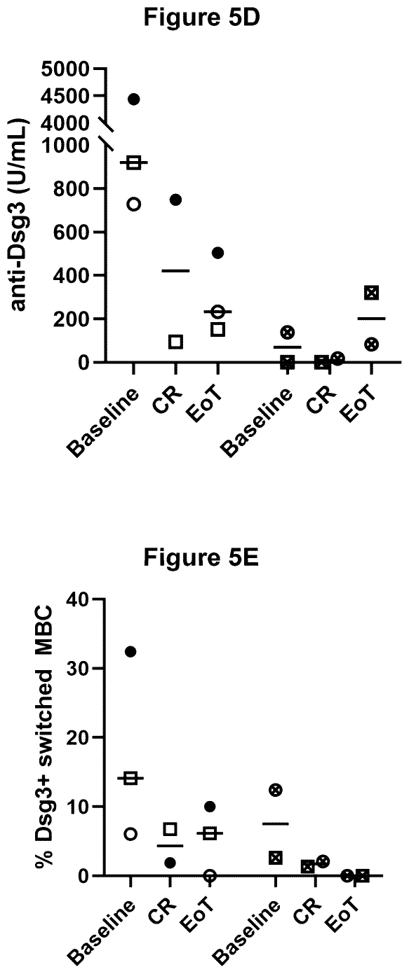

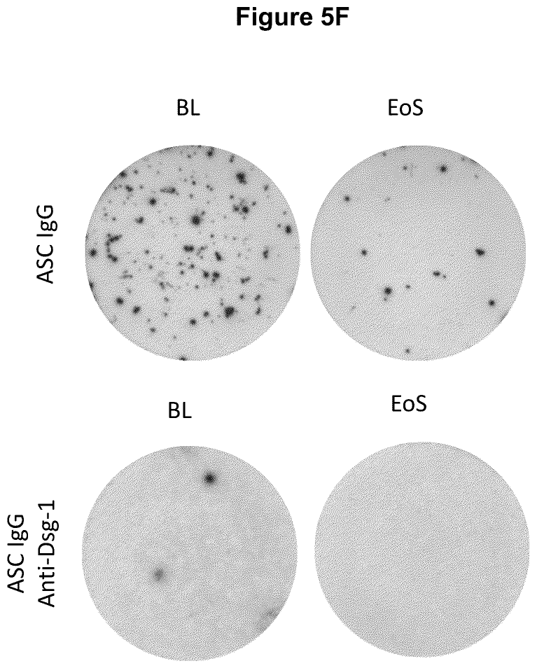

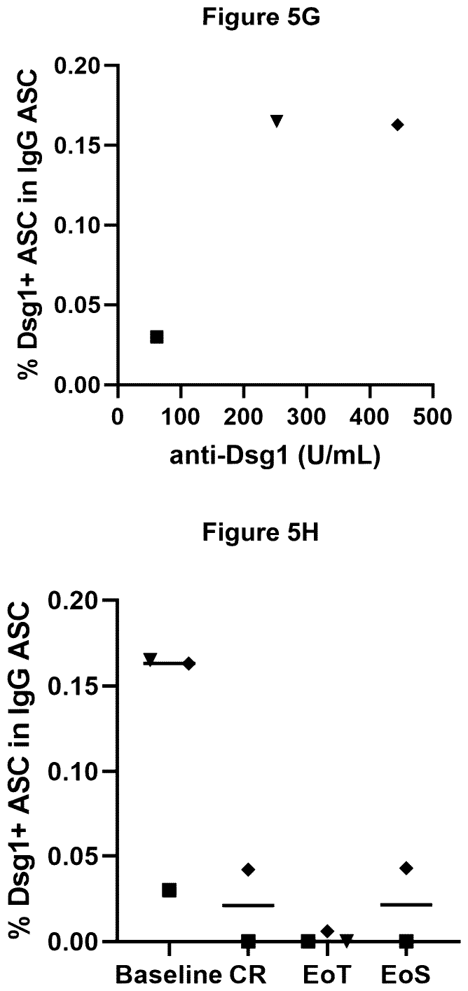

Frequency of Dsg-3+ B cells in periphery was identified by staining for CD45+, CD19+ and CD27+ memory B cells (MBCs) and fluorescently labeled Dsg-3 antigen (FIG. 5A). Additional staining with IgM and IgD was included prior to gating on Dsg-3+ cells to identify class-switched memory B cells (FIG. 1). Dsg-3+ B cells were identified within CD27+ IgM− IgD− cells, known to harbor antigen-specific B cells (FIGS. 5A, B, and C). In line with previous reports, Dsg-3-specific B cells were rarely seen in peripheral blood but were detected principally at baseline and were detected at higher frequencies in patients with higher anti-Dsg-3 serum IgG levels. A reduction of antigen-specific MBCs was visible when patients 5, 6, and 8 reached CR and at end of treatment. Two PV patients that relapsed following CR also exhibited reduction of Ag-specific B cells at CR (FIGS. 5D and 5E) and no increase in frequency of Dsg-3+ B cells was further observed, possibly implicating persistent tissue-resident antigen-specific responses in relapse.

In addition to assessing the frequency of Dsg-3+ B cells in patients with PV, Dsg-1 specific ASCs were measured by ELISPOT. Although ELISPOT assays were performed in all samples, low cell viability after thawing and withdrawal of patient's consent for post hoc analysis restricted data analysis to patients 3, 4, and 7. At baseline, anti-Dsg-1 IgG ASC were detected in all 3 PF patients assessed and were detected at higher frequency in patients with higher serum anti-Dsg1 levels (FIGS. 5F, G, and H). Following treatment with efgartigimod and concomitant low-dose prednisone, anti-Dsg-1 IgG ASC were no longer detectable at end of study. For two patients, disappearance of anti-Dsg-1 IgG ASC was detected at the CR visit (on days 44 and 275, respectively). One patient demonstrated loss of anti-Dsg-1 IgG ASC at end of treatment, while CR was achieved 47 days later during treatment-free follow-up.

### T Cell and B Cell Phenotypes During Efgartigimod Treatment

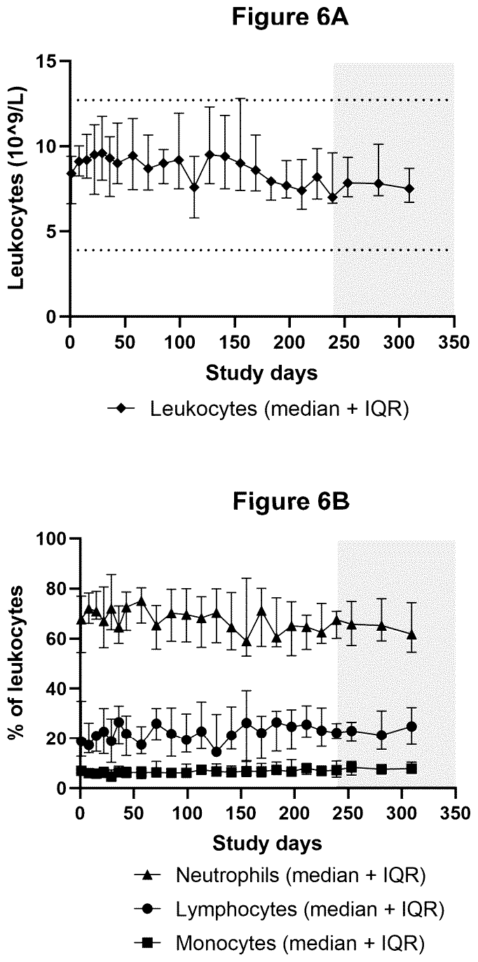

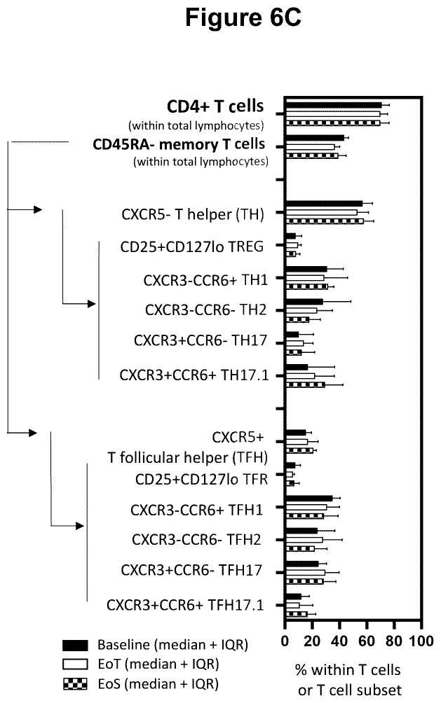

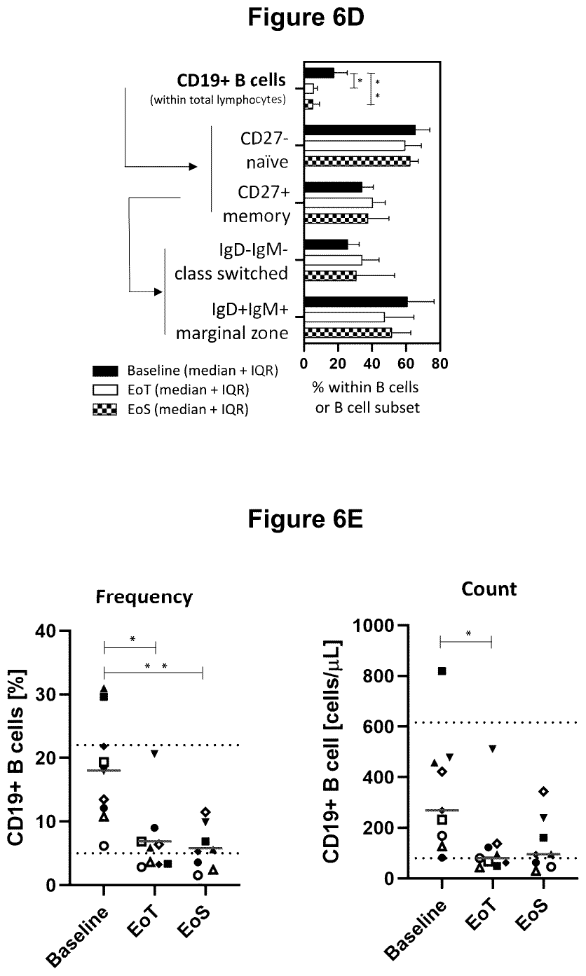

Beyond autoantibody producing B cells, classical type-2 T helper (Th2) cells are typically considered central players in pemphigus pathology; however, recent advances in clinical immunology pointed to additional T cell subsets, namely T helper-17/T follicular helper-17 (Th17/Tfh17) cells efficiently promoting autoantibody production in pemphigus patients. Thus, it was then investigated whether prolonged treatment had an impact on major T cell and B cell subsets in addition to the antigen-specific context. Importantly, treatment did not impact global levels of total leukocytes, total lymphocytes, monocytes, or neutrophils (FIGS. 6A and 6B). Longitudinal analysis of PBMCs in those patients at 4 different time points did not reveal changes in overall T cell subpopulations. Specifically, CD4+ T cells were stable with no significant changes in Th1, Th2, Th17 or Tfh1, Tfh2, Tfh17 subsets (FIG. 6C).

Baseline frequency of total B cells as proportion of total lymphocytes was heterogenous and ranged from 6.2% to 30.9%, indicating high B cell frequency in some individuals, possibly due to B cell activation and proliferation. Surprisingly, analysis of B cells revealed declining numbers of median total CD19+ B cells in the periphery of all nine patients but without affecting the composition of B cell subsets for the markers tested, including CD27+ memory cells (FIGS. 6D and 6E). After treatment, median levels of CD19+ B cells remained within normal ranges.

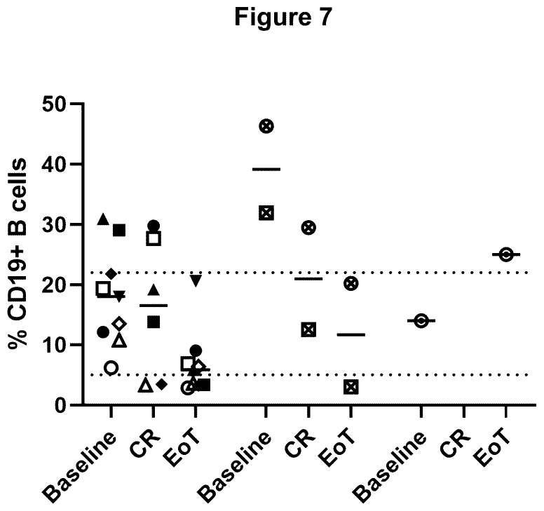

The highest B cell frequencies in the trial were observed in patients 11 and 12, who relapsed to PDAI activity >10 after having achieved CR. At the last evaluable time point, no increases from baseline in B cell frequency were noted. Patient 13 with PF showed no clinical response to efgartigimod treatment and did not demonstrate changes in CD19+ B cells (FIG. 2A and FIG. 7).

Prednisone Exposure in Patients with Prolonged Efgartigimod Treatment

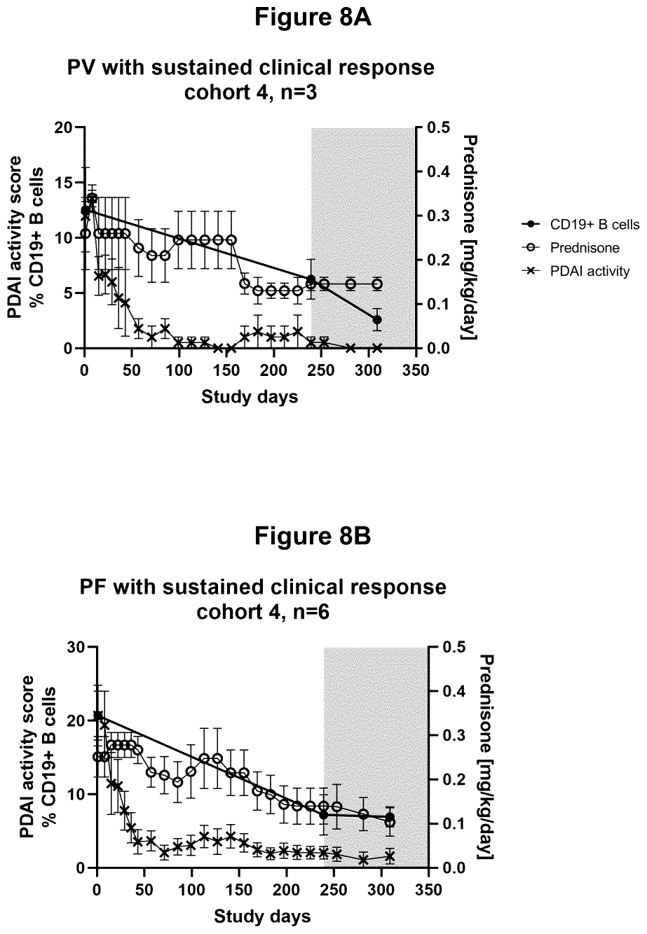

The effect of efgartigimod on the frequency of B cells is likely not due to concomitant treatment with prednisone because B cell levels were not affected in patients in the prednisone-only arm of the RITUX 3 trial in pemphigus (a rituximab clinical trial). Nevertheless, the potential impact of concomitant prednisone exposure on the observed pharmacodynamic and immunological effects described above was analyzed. As seen in the overall study population, early clinical efficacy of efgartigimod allowed for early tapering of steroids. This effect was further pronounced in patients who achieved sustained clinical response after prolonged treatment with efgartigimod in cohort 3 and 4. In patients that achieved CR at any point in the study, the average daily prednisone dose until CR was 0.255 mg/kg/day for cohort 4 patients (n=9) and 0.18 mg/kg/day for cohort 3 patients (n=5) (Table 8), and reduced frequencies of B cells were not associated with higher prednisone dosages (FIG. 8A and FIG. 8B).

TABLE 7
Clinical Status and concomitant prednisone dose levels over time in cohorts 3 and 4.

| | | | | | | | Dose of | | | | | Average |
|---|---|---|---|---|---|---|---|---|---|---|---|---|
| | | | | | | | prednisone | Clinical | | Dose of | Average | daily |
| | | | | | | Delay | at the time | status at | Initial | prednisone | daily | prednisone |
| | | | | | PDAI | to first | of first | the last | dose | at the last | prednisone | dose over |
| | Pemphigus | | | | activity | relapse | relapse | evaluation/ | prednisone | evaluation | dose until | the study |
| | Disease | | | Type of | at | after DC | (mg//kg/ | PDAI | (mg/kg/ | (mg/kg/ | CR (mg/ | (mg/kg/ |
| | History | Gender | Age | Pemphigus | baseline | (days) | day) | activity | day) | day) | kg/day) | day) |
| | Cohort 4 | | | | | | | | | | | |
| 1 | Relapsing | Female | 63 | PF | 9 | | | CR/0 | 0.28 | 0 | 0.273 | 0.080 |
| 2 | Relapsing | Female | 48 | PV M | 27.6 | | | EoC/2 | 0.28 | 0.35 | | 0.278 |
| 3 | Relapsing | Male | 57 | PF | 20.3 | 211 | 0.08 | AD/3 | 0.24 | 0.06 | 0.111 | 0.108 |
| 4 | Newly | Female | 42 | PF | 34.5 | | | CR/0 | 0.31 | 0.11 | 0.218 | 0.207 |
| | diagnosed | | | | | | | | | | | |
| 5 | Relapsing | Female | 62 | PV M | 14.6 | | | CR/0 | 0.37 | 0.13 | 0.370 | 0.194 |
| 6 | Relapsing | Male | 66 | PV MC | 11 | | | DC/1 | 0.10 | 0.10 | | 0.100 |
| 7 | Relapsing | Female | 85 | PF | 11.2 | | | CR/0 | 0.22 | 0.06 | 0.215 | 0.100 |
| 8 | Newly | Male | 47 | PV MC | 10.3 | | | CR/0 | 0.31 | 0.16 | 0.310 | 0.243 |
| | diagnosed | | | | | | | | | | | |
| 9 | Newly | Male | 67 | PF | 19 | | | CR/0 | 0.06 | 0.19 | 0.235 | 0.223 |
| | diagnosed | | | | | | | | | | | |
| 10 | Relapsing | Female | 36 | PF | 30.2 | 63 | 0.20 | EoC/6.3 | 0.40 | 0.20 | | 0.318 |
| 11 | Newly | Male | 58 | PV C | 2 | 200 | 0.23 | AD/27.9 | 0.23 | 1.8 | 0.230 | 0.347 |
| | diagnosed | | | | | | | | | | | |
| 12 | Newly | Female | 66 | PV MC | 7.3 | 82 | 0.08 | AD/10.9 | 0.33 | 0.98 | 0.330 | 0.310 |
| | diagnosed | | | | | | | | | | | |
| 13 | Newly | Female | 51 | PF | 30.3 | | | —/17 | 0.32 | 0.32 | | 0.320 |
| | diagnosed | | | | | | | | | | | |
| 14 | Newly | Male | 22 | PV MC | 39.9 | | | DC/23.2 | 0.33 | 1.3 | | 0.943 |
| | diagnosed | | | | | | | | | | | |
| 15 | Relapsing | Male | 30 | PV C | 28.4 | | | DC/17.8 | 0.61 | 0.61 | | 0.610 |
| | Cohort 3 | | | | | | | | | | | |
| 16 | Newly | Female | 40 | PV M | 2 | | | DC/1 | 0 | 0.06 | 0.027 | 0.071 |
| | diagnosed | | | | | | | | | | | |
| 17 | Relapsing | Male | 36 | PV MC | 3 | | | DC/1 | 0.14 | 0.14 | | 0.134 |
| 18 | Relapsing | Female | 48 | PV M | 12 | 169 | 0.09 | AD/2.6 | 0.54 | 0.09 | | 0.123 |
| 19 | Relapsing | Female | 52 | PV M | 1 | 92 | 0.17 | AD/3.3 | 0.06 | 0.65 | 0.060 | 0.188 |
| 20 | Newly | Female | 65 | PV MC | 23 | | | CR/0 | 0 | 0.27 | 0.053 | 0.226 |
| | Diagnosed | | | | | | | | | | | |
| 21 | Relapsing | Female | 30 | PV MC | 18.9 | | | CR/0 | 0.48 | 0.48 | 0.480 | 0.480 |
| 22 | Relapsing | Female | 54 | PV MC | 14 | 141 | 0.07 | CR/0 | 0.28 | 0.14 | 0.280 | 0.140 |

PV, pemphigus vulgaris;
PF, pemphigus foliaceus;
M, mucosal-dominant;
MC, mucocutaneous;
C, cutaneous;
DC, disease control;
EoC, end of consolidation;
CR, complete clinical remission;
AD, active disease

### C. CONCLUSION

Previous results have shown that treatment of pemphigus patients with efgartigimod results in suppression of IgGs, which demonstrates the strong clinical efficacy of efgartigimod. The results discussed above show for the first time an immunomodulatory effect by an anti-FcRn inhibitor beyond blocking of IgG recycling in pemphigus patients. Specifically, the results demonstrate a prolonged reduction of autoantibody levels during the 10 weeks of efgartigimod-free follow up and a reduction of both antigen specific B cells and changes in the B cell compartment, including a reduction in total CD19+ B cells, after stopping efgartigimod treatment. In contrast, patients who did not respond to efgartigimod treatment did not have a reduced number of CD19+ B cells.

The early clinical improvement of patients allowed for early prednisone tapering, which led to a comparatively lower corticosteroid exposure throughout the study than in the general pemphigus population.

A decrease of CD19+ B cell frequency toward a return to normality (around 10% of lymphocytes) can be used as a biomarker to show regained immune homeostasis and indicate time to discontinue efgartigimod therapy. Further, the effect of efgartigimod on the frequency of B cells in pemphigus patients provides a rationale for using the frequency of B cells in a patient as a marker for monitoring treatment efficacy of an FcRn antagonist in patients with pemphigus and other autoantibody-mediated diseases.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

1. A method for monitoring efficacy of treatment of an autoantibody-mediated disease in a subject following treatment with a first FcRn antagonist, the method comprising:
a) measuring in vitro the frequency of B cells in a blood sample taken from the subject; and
b) comparing the frequency of B cells to a reference value associated with the autoantibody-mediated disease in the subject,
wherein the treatment is not effective if the frequency of B cells in the sample is greater than or equal to the reference value, and wherein the treatment is effective if the frequency of B cells is less than the reference value.

2. A method of treating an autoantibody-mediated disease in a subject that has received a first FcRn antagonist and is receiving a corticosteroid dosing regimen, the method comprising:
a) administering to the subject a therapeutically effective amount of a second FcRn antagonist;
b) measuring in vitro the frequency of B cells in a blood sample taken from the subject; and
c) comparing the frequency of B cells to a reference value associated with the autoantibody-mediated disease in the subject,
wherein the corticosteroid dosing regimen is maintained if the frequency of B cells in the sample is greater than or equal to the reference value, or wherein the corticosteroid dosing regimen is tapered if the frequency of B cells is less than the reference value.

3. A second FcRn antagonist for use in a method of treating an autoantibody-mediated disease in a subject that has received a first FcRn antagonist and is receiving a corticosteroid dosing regimen, wherein:
a) a therapeutically effective amount of the second FcRn antagonist is administered to the subject;
b) the frequency of B cells in a blood sample taken from the subject is measured in vitro; and
c) the frequency of B cells is compared to a reference value associated with the autoantibody-mediated disease in the subject,
wherein the corticosteroid dosing regimen is maintained if the frequency of B cells in the sample is greater than or equal to the reference value, and wherein the corticosteroid dosing regimen is tapered if the frequency of B cells is less than the reference value.

4. A method for treating an autoantibody-mediated disease in a subject comprising:
(a) administering to the subject one or more initial doses of a therapeutically effective amount of a first FcRn antagonist,
(b) administering to the subject one or more further doses of a therapeutically effective amount of a second FcRn antagonist if the frequency of B cells in the subject after step (a) is greater than or equal to a reference value associated with the autoantibody-mediated disease in the subject, or discontinuing treatment with the first FcRn antagonist if the frequency of B cells in the subject after step (a) is less than a reference value associated with active disease in the subject.

5. An FcRn antagonist for use in a method of treating an autoantibody-mediated disease in a subject, wherein
(a) one or more initial doses of a therapeutically effective amount of a first FcRn antagonist is administered to the subject, and
(b) one or more further doses of a therapeutically effective amount of a second FcRn antagonist is administered to the subject if the frequency of B cells in the subject after step (a) is greater than or equal to a reference value associated with the autoantibody-mediated disease in the subject or the first FcRn antagonist is discontinued if the frequency of B cells in the subject after step (a) is less than a reference value associated with the autoantibody-mediated disease in the subject.

6. The method of claim 4 or claim 5, wherein the therapeutically effective amount of the first FcRn antagonist is a dose of about 10 mg/kg to about 30 mg/kg, administered intravenously.

7. The method of claim 4 or claim 5, wherein the therapeutically effective amount of the first FcRn antagonist is a dose of about 750 mg to about 3000 mg, administered subcutaneously.

8. A method for determining if a subject that has previously been treated for an autoantibody-mediated disease using a first FcRn antagonist requires further treatment with a second FcRn antagonist, the method comprising:
a) measuring in vitro the frequency of B cells in a blood sample taken from the subject; and
b) comparing the frequency of B cells to a reference value associated with the autoantibody-mediated disease in the subject,
wherein if the frequency of B cells in the sample is greater than or equal to the reference value, then the subject is in need of further treatment with the second FcRn antagonist.

9. A method for treating an autoantibody-mediated disease in a subject, the method comprising: administering to the subject a therapeutically effective amount of a second FcRn antagonist,
wherein the autoantibody-mediated disease has relapsed in the subject following prior therapy with a first FcRn antagonist and wherein the subject has a frequency of B cells that is greater than or equal to a reference value associated with the autoantibody-mediated disease in the subject.

10. A second FcRn antagonist for use in a method of treating an autoantibody-mediated disease in a subject, wherein the autoantibody-mediated disease has relapsed in the subject following prior therapy with a first FcRn antagonist and wherein the subject has a frequency of B cells that is greater than or equal to a reference value associated with the autoantibody-mediated disease in the subject, and wherein a therapeutically effective amount of the second FcRn antagonist is administered to the subject.

11. A method for treating an autoantibody-mediated disease in a subject, wherein the autoantibody-mediated disease has relapsed in the subject following prior therapy with a first FcRn antagonist, wherein a therapeutically effective amount of a second FcRn antagonist is administered to the subject, and wherein the therapeutically effective amount is determined based on the frequency of B cells in a blood sample taken from the subject.

12. A second FcRn antagonist for use in a method of treating an autoantibody-mediated disease in a subject, wherein the autoantibody-mediated disease has relapsed in the subject following prior therapy with a first FcRn antagonist, wherein a therapeutically effective amount of the second FcRn antagonist is administered to the subject, and wherein the therapeutically effective amount is determined based on the frequency of B cells in a blood sample taken from the subject.

13. The method or use of claim 11 or 12, wherein the frequency of B cells is about 1.5-fold, 2-fold, 3-fold, 4-fold, 5-fold, 6-fold, or 7-fold times a normal frequency of B cells.

14. The method or use of claim 13, wherein the normal frequency of B cells is about 3%, 5%, 10%, 15%, 20%, or 30% of lymphocytes.

15. A method for monitoring remission of an autoantibody-mediated disease in a subject following treatment with a first FcRn antagonist, the method comprising:
a) measuring in vitro the frequency of B cells in a blood sample taken from the subject; and
b) comparing the frequency of B cells to a reference value associated with the autoantibody-mediated disease in the subject,
wherein the subject is in remission from the autoantibody-mediated disease if the frequency of B cells in the sample is lower than the reference value.

16. The method of claim 2 or claim 3, wherein the corticosteroid dose regimen is tapered to a lower dose amount or a lower dosing frequency.

17. The method of claim 8, further comprising administering to the subject a therapeutically effective amount of the second FcRn antagonist if the frequency of B cells in the sample is greater than or equal to the reference value.

18. The method of any one of claim 1 or 15, further comprising administering to the subject a therapeutically effective amount of a second FcRn antagonist if the frequency of B cells in the sample is greater than or equal to the reference value.

19. The method or use of any one of claim 1-10 or 15-18, wherein the reference value is about 1.5-fold, 2-fold, 3-fold, 4-fold, 5-fold, 6-fold, or 7-fold times a normal frequency of B cells.

20. The method or use of claim 19, wherein the normal frequency of B cells is about 3%, 5%, 10%, 15%, 20%, 25%, or 30% of lymphocytes.

21. The method or use of any one of claim 1-10 or 15-18, wherein the reference value is about 3%, 5%, 10%, 15%, 20%, 25%, or 30% of lymphocytes.

22. The method or use of any one of claim 1-10 or 15-18, wherein the reference value is about 10%, 15%, 20%, 25%, 30%, 35%, 40%, 45%, 50%, 55%, 60%, 65%, 70%, 75%, 80%, 85%, or 90% of the maximum frequency of B cells measured in the subject prior to receiving any treatment for the autoantibody-mediated disease.

23. The method or use of any one of claim 1-10 or 15-18, wherein the reference value is greater than 60% of the maximum frequency of B cells measured in the subject prior to receiving any treatment for the autoantibody-mediated disease.

24. The method or use of any one of claim 1-10 or 15-18, wherein the reference value is about 20%, 25%, 30%, 35%, 40%, 45%, 50%, 55%, or 60% of the maximum frequency of B cells measured in the subject prior to being treated with the first FcRn antagonist for the autoantibody-mediated disease.

25. The method or use of any one of claim 1-10 or 15-18, wherein the reference value is at least 60% of the maximum frequency of B cells measured in the subject prior to being treated with the first FcRn antagonist for the autoantibody-mediated disease.

26. The method or use of any one of claim 1-10 or 15-18, wherein the reference value is about 2-fold, 3-fold, 4-fold, 5-fold, or 6-fold times the lowest frequency of B cells measured in the subject following treatment with the first FcRn antagonist for the autoantibody-mediated disease.

27. The method or use of any one of claim 1-3 or 8-26, wherein the subject was previously treated with the first FcRn antagonist at a dose of about 10 mg/kg to about 30 mg/kg, administered intravenously.

28. The method or use of any one of claim 1-3 or 8-26, wherein the subject was previously treated with the first FcRn antagonist at a dose of about 750 mg to about 3000 mg, administered subcutaneously.

29. The method or use of any one of claims 1-28, wherein the subject was previously treated with a corticosteroid or an immunosuppressive agent.

30. The method or use of any one of claim 2-7, 9-14, or 16-18, wherein the effective amount of the second FcRn antagonist is a higher dose than the previous treatment with the first FcRn antagonist.

31. The method or use of any one of claim 2-7, 9-14, or 16-18, wherein the effective amount of the second FcRn antagonist is a lower dose than the previous treatment with the first FcRn antagonist.

32. The method or use of any one of claim 2-7, 9-14, or 16-18, wherein the effective amount of the second FcRn antagonist is administered more frequently compared to the previous treatment with the first FcRn antagonist.

33. The method or use of any one of claim 2-7, 9-14, or 16-18, wherein the effective amount of the second FcRn antagonist is administered less frequently compared to the previous treatment with the first FcRn antagonist.

34. The method or use of any one of claim 2-7, 9-14, or 16-18, wherein the effective amount of the second FcRn antagonist is administered intravenously at a dose of about 10 mg/kg to about 30 mg/kg once weekly, every two weeks, every three weeks, every four weeks, or every six weeks.

35. The method or use of claim 34, wherein the effective amount of the second FcRn antagonist is administered intravenously at a dose of 10 mg/kg once weekly, every two weeks, every three weeks, every four weeks, or every six weeks.

36. The method or use of claim 34, wherein the effective amount of the second FcRn antagonist is administered intravenously at a dose of 25 mg/kg once weekly, every two weeks, every three weeks, every four weeks, or every six weeks.

37. The method or use of any one of claim 2-7, 9-14, or 16-18, wherein the effective amount of the second FcRn antagonist is administered subcutaneously at a fixed dose of about 750 mg to about 3000 mg once weekly, every two weeks, every three weeks, every four weeks, or every six weeks.

38. The method or use of claim 37, wherein the effective amount of the FcRn second antagonist is administered subcutaneously at a fixed dose of 1000 mg or 2000 mg once weekly, every two weeks, every three weeks, every four weeks, or every six weeks.

39. The method or use of any one of claim 1 or 4-18, further comprising administering to the subject an effective amount of a corticosteroid or an immunosuppressive agent.

40. The method or use of claim 39, wherein the effective amount of the corticosteroid is administered at a dose of about 0.5 mg/kg per day.

41. The method or use of claim 39, wherein the effective amount of the corticosteroid is administered at a dose of about 0.25 mg/kg per day.

42. The method or use of claim 39, wherein the effective amount of the corticosteroid is administered at a dose of about 20 mg per day.

43. The method or use of claim 39, wherein the effective amount of the corticosteroid is administered at a dose of about 10 mg per day.

44. The method or use of any one of the preceding claims, wherein the frequency of B cells is measured with flow cytometry.

45. The method or use of any one of the preceding claims, wherein the B cells are CD19+ B cells.

46. The method or use of any one of claim 2-14 or 16-45, wherein the first FcRn antagonist and the second FcRn antagonist are each the same FcRn antagonist.

47. The method or use of any one of claim 2-14 or 14-45, wherein the first FcRn antagonist and the second FcRn antagonist are each a different FcRn antagonist.

48. The method or use of claim 46, wherein the FcRn antagonist is an anti-FcRn antibody.

49. The method or use of claim 47, wherein the first FcRn antagonist is an anti-FcRn antibody.

50. The method or use of claim 47, wherein the second FcRn antagonist is an anti-FcRn antibody.

51. The method or use of any one of claims 48-50, wherein the anti-FcRn antibody is rozanolixizumab (UCB7665), nipocalimab (M281), orilanolimab (ALXN1830/SYNT001), or batoclimab (IMVT-1401/RVT1401/HBM9161).

52. The method or use of claim 46, wherein the FcRn antagonist is an Fc region comprising amino acids Y, T, E, K, F, and Y at EU positions 252, 254, 256, 433, 434, and 436, respectively.

53. The method or use of claim 47, wherein the first FcRn antagonist or the second FcRn antagonist is an Fc region comprising amino acids Y, T, E, K, F, and Y at EU positions 252, 254, 256, 433, 434, and 436, respectively.

54. The method or use of claim 46, wherein the FcRn antagonist is efgartigimod.

55. The method or use of claim 47, wherein the first FcRn antagonist or the second FcRn antagonist is efgartigimod.

56. The method or use of claim 46, wherein the FcRn antagonist comprises the amino acid sequence of SEQ ID NO: 1, 2, or 3.

57. The method or use of claim 47, wherein the first FcRn antagonist or the second FcRn antagonist comprises the amino acid sequence of SEQ ID NO: 1, 2, or 3.

58. The method or use of claim 47, wherein the first FcRn antagonist is an anti-FcRn antibody and the second FcRn antagonist is efgartigimod.

59. The method or use of claim 47, wherein the first FcRn antagonist is an anti-FcRn antibody and the second FcRn antagonist comprises the amino acid sequence of SEQ ID NO: 1, 2, or 3.

60. The method or use of claim 58 or 59, wherein the anti-FcRn antibody is rozanolixizumab (UCB7665), nipocalimab (M281), orilanolimab (ALXN1830/SYNT001), or batoclimab (IMVT-1401/RVT1401/HBM9161).

61. The method or use of any one of claims 1-51, wherein the patient has not been previously treated with efgartigimod.

62. The method of any one of the preceding claims, wherein the subject has one or more physical symptoms of an autoantibody-mediated disease following treatment with the first FcRn antagonist.

63. The method of any one of the preceding claims, wherein the subject has a serum level of a pathogenic IgG autoantibody that is associated with relapse of the autoantibody-mediated disease.

64. The method of claim 63, wherein the pathogenic IgG autoantibody is an anti-Dsg-3 antibody or an anti-Dsg-1 antibody.

65. The method or use of any one of the preceding claims, wherein the autoantibody-mediated disease is selected from the group consisting of: allogenic islet graft rejection, alopecia areata, ankylosing spondylitis, antiphospholipid syndrome (APS), autoimmune Addison's disease, Alzheimer's disease, antibody-mediated allograft rejection (AMR), antineutrophil cytoplasmic autoantibodies (ANCA), ANCA vasculitis, autoimmune diseases of the adrenal gland, autoimmune encephalitis, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenia purpura or idiopathic thrombocytopenia purpura or immune-mediated thrombocytopenia), autoimmune urticaria, Behcet's disease, bullous pemphigoid (BP), cardiomyopathy, Castleman's syndrome, celiac spruce-dermatitis, chronic fatigue immune disfunction syndrome, chronic inflammatory demyelinating polyneuropathy (CIDP), Churg-Strauss syndrome, cicatricial pemphigoid, CREST syndrome, cold agglutinin disease, COVID-19 mediated postural orthostatic tachycardia syndrome (POTS), Crohn's disease, delayed graft function after kidney transplant, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre syndrome (GBS), Goodpasture's syndrome, graft-versus-host disease (GVHD), Hashimoto's thyroiditis, hemophilia A, hemolytic disease of the fetus and newborn (HDFN), idiopathic membranous neuropathy, idiopathic pulmonary fibrosis, IgA neuropathy, IgM polyneuropathies, juvenile arthritis, Kawasaki's disease, lichen planus, lichen sclerosus, lupus erythematosus, lupus nephritis, membranous neuropathy, membranous nephropathy, Ménière's disease, mixed connective tissue disease, mucous membrane pemphigoid, multiple sclerosis, Type 1 diabetes mellitus, multifocal motor neuropathy (MMN), myelin oligodendrocyte glycoprotein antibody-associated disease (MOGAD), myasthenia gravis (MG), generalized myasthenia gravis (gMG), ocular myasthenia gravis (OMG), myositis, neuromyelitis optica (NMO), paraneoplastic bullous pemphigoid, pemphigoid gestationis, pemphigus vulgaris (PV), pemphigus foliaceus (PF), pernicious anemia, polyarteritis nodosa, polychrondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Raynaud's phenomenon, Reiter's syndrome, rheumatoid arthritis (RA), sarcoidosis, scleroderma, Sjögren's syndrome, solid organ transplant rejection, stiff-man syndrome, systemic lupus erythematosus (SLE), Takayasu's arteritis, toxic epidermal necrolysis (TEN), Stevens-Johnson syndrome (SJS), temporal arteritis/giant cell arteritis, thrombotic thrombocytopenia purpura (TTP), thyroid eye disease, ulcerative colitis, uveitis, warm autoimmune hemolytic anemia (wAIHA), dermatitis herpetiformis vasculitis, anti-neutrophil cytoplasmic antibody-associated vasculitides, vitiligo, and Wegner's granulomatosis.

66. The method of any one of claims 1-64, wherein the autoantibody-mediated disease is pemphigus vulgaris (PV) or pemphigus foliaceus (PF).
